(19) United States
(12) Patent Application Publication
(10) Pub. No.: US 2023/0077323 A1
(43) Pub. Date: Mar. 16, 2023

(54) A RADAR SYSTEM FOR DYNAMICALLY MONITORING AND GUIDING ONGOING CLINICAL TRIALS

(71) Applicant: BRIGHT CLINICAL RESEARCH LIMITED, Hong Kong (CN)

(72) Inventor: Tailiang XIE, Belle Mead, NJ (US)

(21) Appl. No.: 17/797,062

(22) PCT Filed: Feb. 26, 2021
(86) PCT No.: PCT/IB2021/051634

§ 371 Date: Aug. 2, 2022

Related U.S. Application Data
(60) Provisional application No. 63/138,422, filed on Jan. 16, 2021.
(60) Provisional application No. 63/058,839, filed on Jul. 30, 2020.
(60) Provisional application No. 63/016,572, filed on Apr. 28, 2020.
(60) Provisional application No. 62/981,954, filed on Feb. 26, 2020.

(51) Int. Cl. G06F 3/04847; G16H 10/20

(52) U.S. Cl. CPC G06F 3/04847; G16H 10/20

(57) ABSTRACT

The present invention constructs a “radar” system for dynamically monitoring and guiding ongoing clinical trials. In one embodiment, the system partitions the data space into 3 primary regions comprising “favorable”, “hopeful” and “undesirable” to reflect the trial status. In one embodiment, the undesirable region comprises a futility region, and the favorable region comprises a successful region. In one embodiment, the boundaries defining these regions are subject to adjustment as the clinical trial proceeds. In one embodiment, the accumulative treatment effect, data trends, stopping boundaries, trajectory and other information are graphically displayed on the “radar” screen. In one embodiment, the system takes learning from the observed and accumulated data and performs simulations to intelligently guide the trials. In one embodiment, the system is used in re-analysis or diagnosis of clinical trials already completed and provides guidance for clinical trial design or amendment.

### CROSS-REFERENCE TO RELATED APPLICATIONS

This application claims the benefit of U.S. Ser. No. 63/138,422, filed Jan. 16, 2021, U.S. Ser. No. 63/058,839, filed Jul. 30, 2020, U.S. Ser. No. 63/016,572, filed Apr. 28, 2020 and U.S. Ser. No. 62/981,954, filed Feb. 26, 2020. The entire contents and disclosures of the preceding applications are incorporated by reference into this application. Throughout this application, various publications are cited. The disclosures of these publications in their entireties are hereby incorporated by reference into this application to more fully describe the state of the art to which this invention pertains.

### FIELD OF THE INVENTION

This invention relates to systems and associated methods for monitoring of ongoing clinical trials on a dynamic and adjustable fashion, called dynamic data monitoring (DDM). Specifically, the present invention constructs a clinical trial “radar screen” by partitioning the data space into three primary regions: “favorable region”, “hopeful region” and “undesirable region”. The “undesirable region” is further partitioned into “undesirable” and “futility” regions. On the screen, the accumulative treatment effect, data trends, stopping boundaries, trajectory and other information are dynamically and graphically displayed. As a metaphor, the ongoing clinical trial is like an airplane flying in the sky, the accumulative treatment effect is like the trace of travel, the regions indicate the air or weather conditions in the sky, the Independent Data Monitoring Committee (IDMC) plays the role like a ground controller, the “destination” is where the treatment effect crosses the success boundary at the time when study is complete (i.e. statistical significance achieved).

### BACKGROUND OF THE INVENTION

It was reported that 69.3% of Phase II clinical trials have failed to reach Phase III [1]. The high failure rate may have many causes including ineffectiveness of the experimental treatment itself or safety issues. Another cause may be related to deficiencies or limitations of traditional study design. When designing a clinical trial, people typically assume the expected treatment effect based on prior knowledge on the experimental therapy from early phase studies. The assumed treatment effect is used to determine the initial sample size (No), the initial maximum information. As the trial on-going, the information fraction is defined as the proportion of enrolled patients (n) over the No, denoted by t=n/N₀. The challenge is that such estimates from prior or external source may not be reliable because of perhaps different patient populations or medical procedures. Thus, the prefixed maximum information in general, or sample size in specific, may not provide the desired power. An overly optimistic assumed treatment effect will result in insufficient statistical power (or too low in sample size), whereas a pessimistic treatment effect will result in unnecessarily large study. A fixed sample size (SS) may lead to a situation that the trial is hopeful but short for being statistically significant, or that the trial is “hopeless” at an early time but unconsciously carried on to its end without knowing the bad situation. Most of clinical trials are randomized and double-blinded. Thus, patients, trial investigators (physicians) and trial sponsors or other parties of interest may not be aware of the risk or benefit because they have no access to the ongoing clinical trial.

The traditional fixed sample size design is still commonly used in clinical trials, especially for early phase studies, developments in the past decades in trial design have aimed to improve the efficiency of trials. One of the most widely used is the Group Sequential Design (GSD), especially long-term studies. In the classical GSD, interim analyses are conducted at pre-defined time-points with pre-determined thresholds for efficacy or futility (Pocock, 1977 [2]; O'Brien and Fleming (OBF), 1979 [3]; Tsiatis, 1982 [4]). The classical GSD was much enhanced by the alpha-spending function approach (Lan and DeMets, 1983 [5]; Lan and Wittes, 1988 [6]; Lan and DeMets, 1989 [7]; Lan, Rosenberger, and Lachin, 1993 [8]) with flexible analysis schedules and frequencies during the trial. The sample size recalculation (SSR) procedure based on Conditional Power (CP), developed in the early 90's by utilizing the interim data of the current trial itself, aims to secure the study power through possibly increasing the maximum information originally specified in the protocol (Wittes and Brittain, 1990 [9]; Shih, 1992 [10]; Gould and Shih, 1992 [11]; Herson and Wittes, 1993 [12]). See a commentary on GSD and SSR by Shih (2001) [13]. The GSD with SSR formed the so-called adaptive GSD (AGSD) (Bauer and Kohne (1994) [14], Proschan and Hunsberger (1995) [15], Cui, Hung and Wang (1999) [16], Li et al. (2002) [17], Chen, DeMets and Lan (2004) [18], Posch et al. (2005) [19], Gao, Ware and Mehta (2008) [20], Gao, Liu and Mehta (2013) [21], Bowden and Mander (2014) [22], and Shih, Li and Wang (2016) [23]). Both GSD and AGSD are commonly used for improving the efficiency of trials. However, there are some limitations and challenges as follows.

First, the timing of interim analysis and/or SSR is pre-defined. Conventionally, practitioners frequently suggested half-way through the trial. Due to the fluctuation of accumulated data, the mid-trial time may be a wrong spot and the interim analysis at it may not reflect the true status (trend) of the data as illustrated in the following table with two extreme scenarios.

TABLE 1
Extreme scenarios with pre-planned interim analyses

| True | SS based | Assumed | SS based on | 50% of | |
|---|---|---|---|---|---|
| δ | on true | δ | assumed | planned SS | Comment |
| 0.2 | 526 | 0.4 | 133 | 67 | Too early |
| 0.4 | 133 | 0.2 | 526 | 263 | Too late |

δ is the treatment effect, 90% power and assume σ = 1.

Second, many Phase II-III clinical trials have an Independent Data Monitoring Committee (IDMC) formed for periodically reviewing the safety and/or efficacy data of the trial as it is on-going. The IDMC usually meets every 3 or 6 months depending on the disease and specific intervention. For an oncology trial with new regimen, the IDMC may want to meet more frequently than a trial for non-life-threatening disease. The committee may want to meet more frequently at the early stage of the trial to understand the safety profile sooner. The current practice for IDMC involves three parties: Sponsor, Independent Statistical Group (ISG) and IDMC. The sponsor's responsibility is to conduct and manage the on-going study. The ISG prepares blinded and unblinded data packages: tables, listing and figures (TLFs) based on the scheduled data cut (usually more than a month before the IDMC meeting). The preparation work usually takes about 2-3 months. The IDMC members receive the data packages a week before the IDMC meeting and will review it during the meeting.

Current IDMC practice has practical problems. First, the data package presented is only a “snapshot” of the data. In other words, the trend of treatment effect (efficacy or safety) as the data accumulate is not presented to IDMC. IDMC's recommendation based on a snapshot may differ from that based on a “continuous” trace of data as illustrated in the following plots.

As shown in FIG. 1A, IDMC may recommend both trials to continue at interim 1 and 2, whereas in FIG. 1B, the negative trend may lead to IDMC to recommend terminating trial B. Second, the current IDMC process has a logistic issue. It takes about 2-3 months for ISG to prepare the data package for IDMC. For a blinded study, the unblinding is usually handled by the ISG. Although it is assumed that the data integrity will be preserved at the ISG level, it is not 100% warranted in this human-handling process without any human errors.

Third, the statistical theories for GSD/AGSD assumed Brownian motion model on the data observed, which induces a linear trend for the data observed (Proschan, Lan, and Wittes, 2006) [24]). In actuality, this assumption might be violated due to some known or unknown reasons such as operational learning curve, changes in the protocol or patients, etc. Once the assumption violated, the statistical tests, models, predictions and the conclusions may not be valid anymore.

FIG. 2 illustrates a data history displayed by B-values B(t), as defined in Lan and Wittes (1988) [6] associated with a regular and asymptotically linear (RAL) test statistic referred in Scharfstein et al. (1997) ([40], versus the information fraction t for a study up to an interim analysis at t=0.75. B (t)=Z(t)V, where Z(t) is the Z-test based on the RAL statistic. Under the model of Brownian motion, we expect to see a linear trend of B(t). In this graph, however, one may suspect that three pieces of linear trend could fit better than just one linear trend. This visual examination is not a formal diagnostic test. However, the whole history of data up to the time of an interim analysis obviously helps to suggest performing some sensitivity analysis at t=0.75.

To be specific, we start with the following well-known result of CP given in, for example, Proschan, Lan and Wittes (2006) [24]. Let the final critical value for B(1) be C_{α}, which equals 1.96 for α=0.025 when no multiplicity adjustment is involved. The CP at the information time t conditioning on B(t) is given by

$$C P ( θ , t ) = P ( B ( 1 ) \geq {C}_{α} \vert B ( t ) ) = 1 - Φ ( \frac{{C}_{α} - B ( t ) - θ ( 1 - t )}{\sqrt{1 - t}} ) , \qquad ( 1 )$$

where θ is the drift parameter which represents the true (unknown) treatment effect in terms of the B-value. There are many ways of choosing θ in (1). Choice depends on the monitoring objectives, such as the specific value in the alternative hypothesis H_{A} on which the original sample size and power were based; 0 under H₀; empirical point estimate

$$\hat{θ} = \frac{B ( t )}{t} ;$$

some confidence limits based on \hat{θ}; or some combination of the above, perhaps even with other external information or opinion of a clinical meaningful effect that needs to be detected, etc. Moreover, the predictive power is obtained by averaging CP(θ, t) over a prior distribution of θ. The DDM offers all these options. The most popular choice in the literature is

$$\hat{θ} = \frac{B ( t )}{t} ,$$

which is a “snap-shot” of the data at t.

When a graph like FIG. 2 indicates that a piecewise linear trend rather than one slope fits better for the data path, we may want to perform some sensitivity analysis on the CP by considering other choices of θ. For example, FIG. 2 shows 3 segments with slope

$${S}_{1} = \frac{B ( {t}_{1} )}{{t}_{1}} , {S}_{2} = \frac{B ( {t}_{2} ) - B ( {t}_{1} )}{( {t}_{2} - {t}_{1} )} , {S}_{3} = \frac{B ( {t}_{3} ) - B ( {t}_{2} )}{( {t}_{3} - {t}_{2} )} ,$$

respectively, for time period (0, t₁), (t₁, t₂), and (t₂, t₃). A weighted average w₁S₁+w₂S₂+w₃S₃ may be used. It is often reasonable to down weigh the earlier trend, judged by the data maturity and/or nature of treatment effect. Notice that

$$\hat{θ} = \frac{B ( t )}{t}$$

is also a weighted average with the weights proportional to the length of the segment

$$( {w}_{1} = \frac{{t}_{1}}{{t}_{3}} , {w}_{2} = \frac{{t}_{2} - {t}_{1}}{{t}_{3}} , {w}_{3} = \frac{{t}_{3} - {t}_{2}}{{t}_{3}} )$$

instead of the time order. When performing multiple interim analyses, the weights change and this approach becomes a moving (weighted) average for calculating the CP from time to time, with the whole up-to-date data path rather than a “snap-shot” at each time. In DDM, we recommend this approach when the data seem to exhibit non-linear drift.

As pointed out earlier, most of clinical trials are randomized and double-blinded. Thus, patients, trial investigators (physicians) and trial sponsors or other parties of interest may not be aware of the risk or benefit because they have no access to the clinical trial. In one embodiment, the radar system of the present invention is featured for automatically unblinding data without human involvement and continuously evaluating risks based on the unblinded data.

Most clinical trials nowadays are managed by an Electronic Data Capture (EDC) system. Treatment assignment and drug dispensing are managed by an Interactive Responsive Technology (IRT) system. By integrating EDC and IRT together, treatment effect on endpoints of interest (safety or efficacy) can be computed automatically and continuously. This automation enables us to develop a computer system for dynamically monitoring on-going trials and intelligently predicting the trajectory of trial results.

This invention constructs a clinical trial “radar” system for dynamically monitoring and guiding ongoing trials, in which:
(1) The accumulative treatment effect and associated statistics (the CP, sample size ratio, etc.) can be computed automatically.
(2) The model linearity can be assessed automatically.
(3) The data trend and trajectory can be dynamically estimated
(4) Simulations can be performed for assessing the reliability of the estimated trend and trajectory.
(5) Decision can be made intelligently.

In one embodiment, the present invention provides a computer-based “radar” system for clinical trials on which the data space is partitioned into four regions: favorable, hopeful, undesirable and futility, as shown in FIGS. 3A and 3B. When trial data (the accumulated treatment effect) “travels” in the favorable region, the trial is in a good status as expected. When trial data “travels” in the hopeful region, the trial is promising, but not good enough, more samples may be needed. Sample size will be automatically re-estimated. When trial data “travels” in the undesirable region, the trial is not yet deemed to be futile; a weak trend, however, may require unfordable effort (unaffordable sample size) for such clinical trial to reach success; When trial data “travels” in the futility region, the trial is for sure to be futile and can be terminated for avoiding unethical patient suffering and unnecessary financial waste.

### SUMMARY OF THE INVENTION

In one aspect, the present invention provides a computer-based radar system and method for monitoring and guiding an ongoing clinical trial on an adjustable and dynamic basis.

In one embodiment, the radar system comprises a clinical trial database, a treatment database, a dynamic trial design (DTD) module, a dynamic data monitoring (DDM) engine, a trial simulation engine, a parameter input interface and a trial radar display screen. In one embodiment, a graphical user interface encompasses a parameter input interface and a display screen. In one embodiment, the clinical trial database stores patient information from an ongoing clinical trial, wherein said information comprises a set of subject data that is being continuously updated as said ongoing clinical trial proceeds. In one embodiment, the treatment database stores patient's treatment assignment (usually randomly assigned). In one embodiment, the clinical trial database and treatment database are integrated systematically. In one embodiment, the DTD module, based on initial design parameters, partitions the trial data space into four regions: favorable, hopeful and undesirable (alternatively, unfavorable) and futility regions. In one embodiment, the boundaries for these regions are subject to further adjustment when the assumption is modified or during clinical trial. The design parameters usually include, but not limited to the following: hypothesized treatment effect, overall statistical power required, maximal sample size to be willing to take, whether to consider early stopping for efficacy or futility. The boundaries which create the regions are calculated by the initial design parameters. In one embodiment, the DDM engine performs a series of user-specified tasks as patient data accumulated. The tasks include, but not limited to the following:
a. Compute the accumulative treatment effect (efficacy or safety).
b. Compute the CP based on user-chosen hypotheses.
c. Compute the sample size ratio (R)based on current trend over the initial sample size N₀.
d. Assess the linearity of the accumulative treatment effect data.
e. Modify the initial assumption if necessary.
f Update the regions and boundaries according to modified assumption.
g. Compute the “weighted” trend score of the accumulative patient data.
h. Estimate the “weighted” treatment trajectory based on the accumulative patient data.

In one embodiment, the simulation engine performs simulations (at least 1000 times) by adjusting variety of parameters to assess the reliability (or the confidence interval) of the trend and trajectory. In one embodiment, the trial radar screen displays the four regions, stopping boundaries, accumulative treatment effect (efficacy or safety), trend and treatment trajectory. In one embodiment, the DDM engine performs the said tasks on specific patient subgroups.

### Definition and Abbreviations

| # | Abbreviation | Full Name, and Calculation |
|---|---|---|
| 1. | RAL | Regular and asymptotically linear |
| 2. | CP | Conditional power |
| 3. | DTD | Dynamic Trial Design |
| 4. | DDM | Dynamic Data Monitoring |
| 5. | DMC | Data Monitoring Committee |
| 6. | SS | Sample size |
| 7. | R | Sample size ratio R = N_{new}/N₀ |
| 8. | Rₘₐₓ | The maximum sample size ratio to be considered |
| 9. | SSR | Sample size recalculation |
| 10. | Z-score(s) | Standardized efficacy score(s) |
| 11. | EMR | Electronic Medical Records |
| 12. | θ | Treatment effect size |
| 13. | N₀ | A planned initial sample size (or “information” in general) N₀ |
| 14. | α | Type I error |
| 15. | β | Type II error |
| 16. | t | Information fraction time. generally, t = n/N₀, where n is the number of |
| | | patients who reached the study endpoints. Therefore, 0 ≤ t ≤ 1. |

In one embodiment, the present invention provides decision-making guidance as following: Let tₖ and dₖ be the k^{th} interim analysis time and stopping boundary, respectively, k=1, 2, . . . K=final. A guidance for monitoring on-going trials in DDM, with the understanding that K is only for planning purpose, not a fixed number.
(1) Stop the trial early for benefit if Z(tₖ)≥dₖ or B(tₖ)≥dₖ\sqrt{tₖ};
(2) If B(tₖ) falls in the “futility” region persistently, we could consider stopping the trial for futility. However, the decision of futility is non-binding.
(3) In between (1) and (2) above, consider SSR or continue monitoring without any change:
   a) No change if CP(tₖ)≥1−β or equivalently B(tₖ)≥(Z_{β}\sqrt{1−tₖ}+C_{α})t, but not exceeds dₖ\sqrt{tₖ}, i.e., in the “favorable” region;
   b) If γ(tₖ, Rₘₐₓ)≤CP(tₖ)<1−β (i.e. B(tₖ) falls in the “hopeful” region), the trial should continue. If we observe consecutive m (say, 10) points in this region, then re-estimate the SS. Choice of Rₘₐₓ depends on sponsor's affordability. For example, set Rₘₐₓ=3 if it is acceptable. When SS is increased, the future boundary values are re-calculated as well.
   c) If B(tₖ) falls in the “undesirable” region, we would take no decision/action but continue monitoring. If B(t) stays in this region persistently, the trial may be recommended for termination for administrative reason such as exceeding the affordable budget.

### BRIEF DESCRIPTION OF THE FIGURES

FIGS. 1A and 1B show snapshots of the Wald Statistics at interim analysis and continuous display of data, respectively.

FIG. 2 is a display of the nonlinear trend of data.

FIG. 3A is an illustration of radar system in Z-value versus information fraction dividing the trial data space into four regions, i.e., favorable, hopeful (promising), unfavorable (undesirable), and futility regions. FIG. 3B is an illustration of radar system in B-value versus information fraction dividing the trial data space into four regions, i.e., favorable, hopeful (promising), unfavorable (undesirable), and futility regions.

FIG. 4A shows a representative system comprising a clinical trial database, a processing unit and a decision-making unit, wherein the processing unit includes a decryption module, a simulation module, and a statistic module. FIG. 4B illustrates a typical system comprising DTD, DDM and simulation engine and how they interact with database. FIG. 4C illustrates creating of boundaries by DTD module based on design parameters. FIG. 4D illustrates monitoring of data as an on-going clinical trial goes. FIG. 4E shows use of simulation in monitoring. FIG. 4F is a typical workflow showing how a clinical trial is dynamically monitored and how a recommendation to the clinical trial is made. FIG. 4G shows a typical radar system comprising a boundary determination module, a boundary adjustment module and a display module. FIG. 4H is a representative graphical user interface (GUI) with adjustable boundaries.

FIG. 5A shows borderlines for favorable and hopeful regions. FIG. 5B shows lower bound of CP inside hopeful region. As shown, the larger the Rₘₐₓ is, the lower the borderline of “hopeful” region will be or the larger the “hopeful” region will be.

FIGS. 6A and 6B show the Z-value and B-value of treatment effect being monitored on Day 28 as patients accumulated on the radar screen, respectively. FIG. 6C shows CP being monitored on Day 28 as patients accumulated on DDM's radar screen.

FIG. 7A is the Z value and B value being monitored by retrospectively applying DDM to the real positive clinical trial in Example 2. FIG. 7B is the CP being monitored for the real positive clinical trial in Example 2.

FIGS. 8A and 8B are the Z value and B value being monitored by retrospectively applying DDM to the real negative clinical trial in Example 3, respectively.

FIG. 9 shows patients' response rate to placebo (left) and remdesivir (right) in a real clinical trial.

FIG. 10A shows a representative graphical user interface (GUI) at parameter design stage by a DTD module. FIG. 10B is a typical table summarizing all parameters from the GUI for dynamic design. FIG. 10C (left panel) illustrates three (3) regions according to the boundary parameters and a plot based on simulations. FIG. 10C (right panel) shows a predicating result of early efficacy boundary.

FIG. 11A shows a representative GUI for dynamic monitoring during clinical trial. FIG. 11B illustrates panel for connection and communication with patient data. FIG. 11C shows a typical table summarizing all parameters from the GUI for dynamic monitoring. FIG. 11D shows three regions according to the boundary parameters and a plot based on patient data as accumulated.

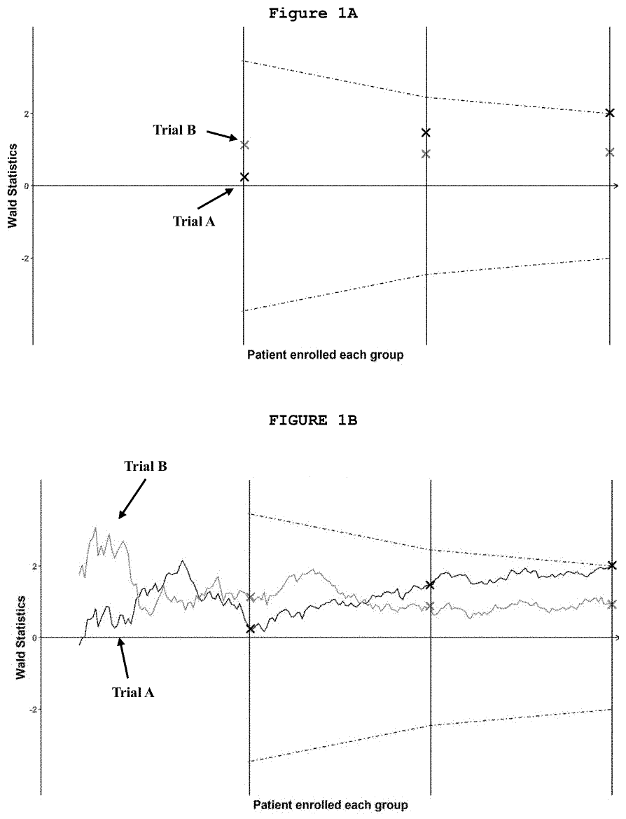

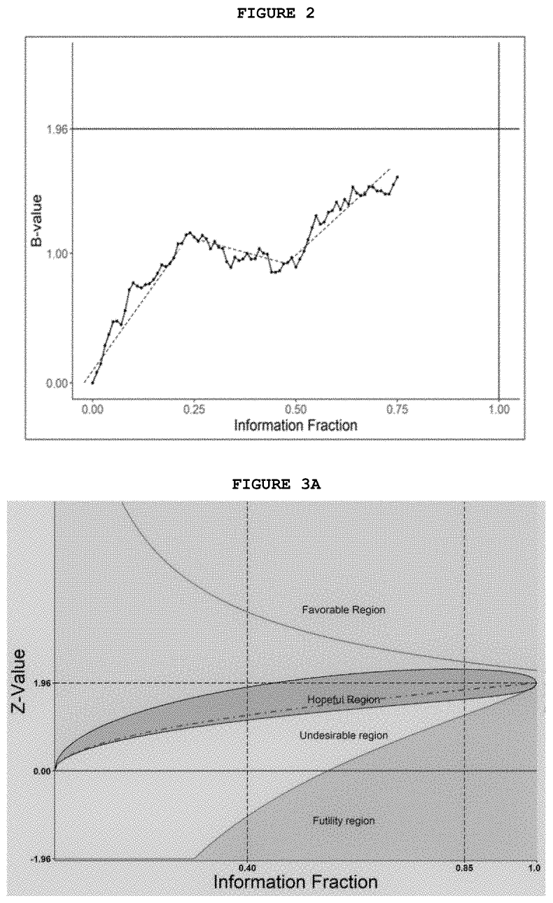

### DETAILED DESCRIPTION OF THE INVENTION

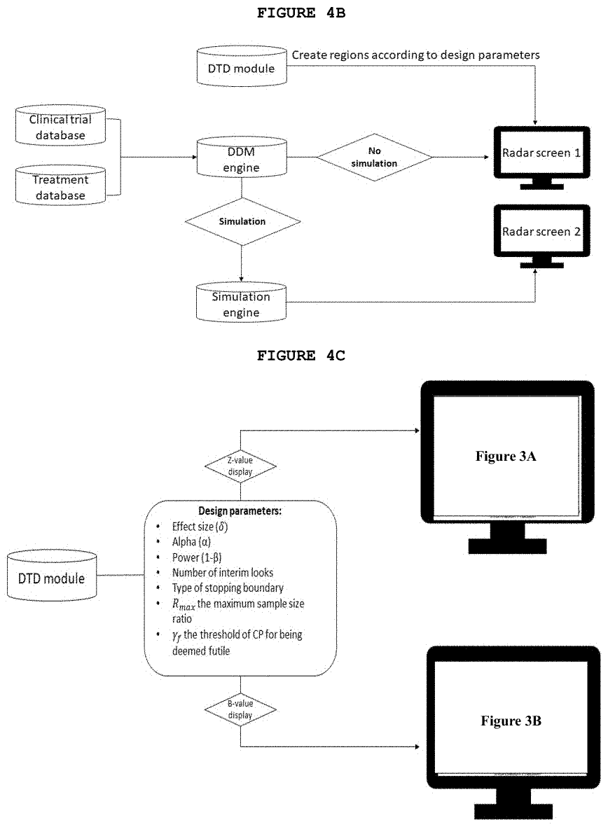

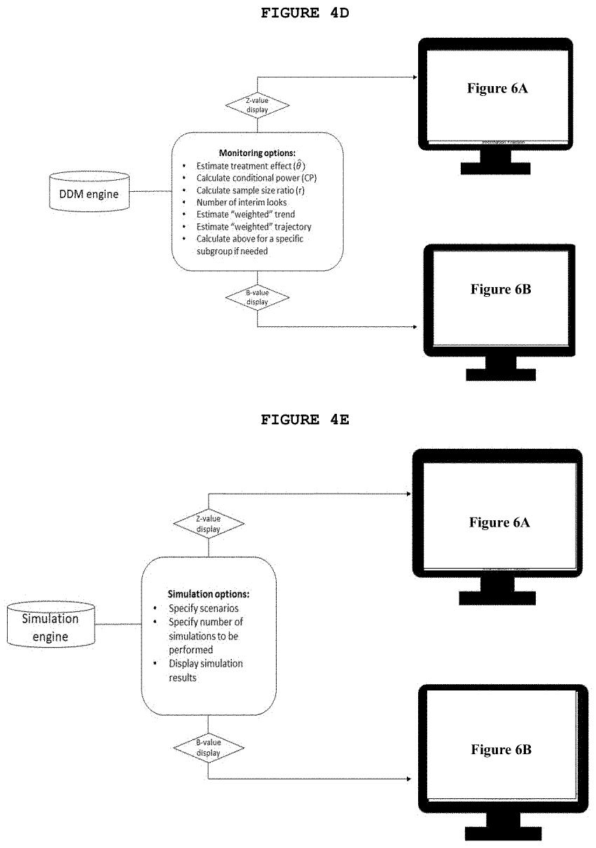

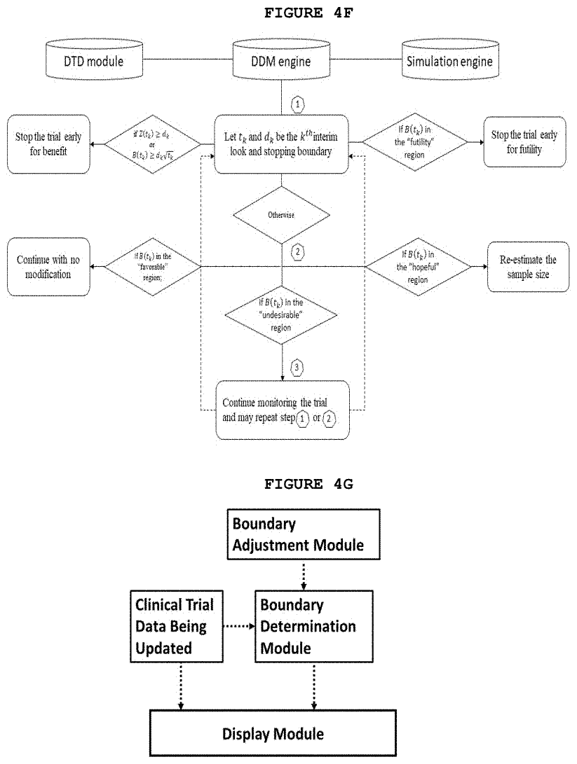

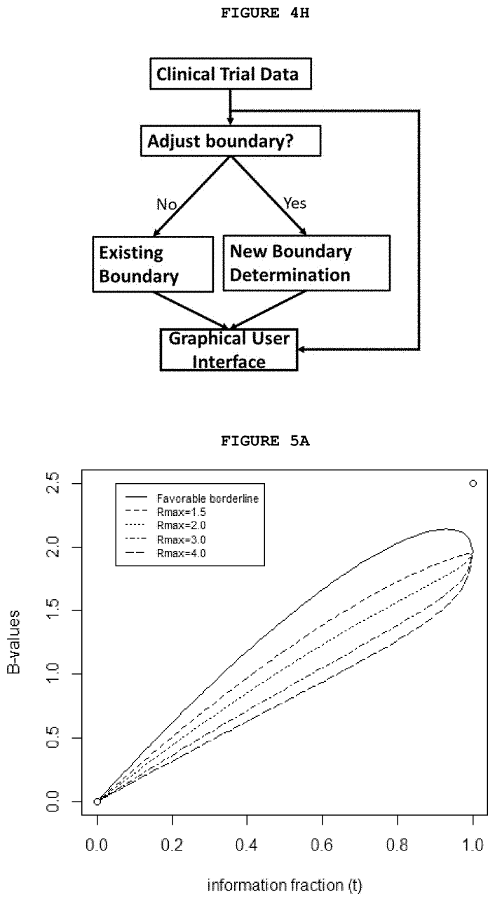

In one aspect, the present invention provides a decision-making system to manage or monitor an ongoing clinical trial. In one embodiment, as shown in FIG. 4A, the system comprises: 1) a clinical trial database for storing information related to said ongoing clinical trial, 2) a processing unit coupled with the clinical trial database, and 3) a decision-making unit.

In one embodiment, the information comprises a set of subjects-data that is encrypted and continuously updated, wherein said set of subjects data comprises a set of control group data and a set of experimental group data. In one embodiment, the processing unit comprises a) a decryption module for decrypting said set of subjects data to identify said set of experimental group data; b) a simulation module for generating a set of simulation data based on said set of experimental group data; and c) a statistic module for computing one or more scores reflecting probability of said on-going double-blind clinical trial being successful, wherein said one or more scores is computed based on said set of experimental group data or said set of simulation data and a set of criteria selected from the group consisting of a favorable criterion, an undesirable criterion, and a promising criterion.

In one embodiment, the decision-making unit is coupled with the clinical trial database and decision-making unit comprises a) a score module to display said one or more scores associated with the on-going double-blind clinical trial; and b) an option module to display one or more options for said user to manage said on-going clinical trial, wherein said one or more options will be feedback to said simulation module to adjust said set of simulated data or said set of criteria and update said one or more scores.

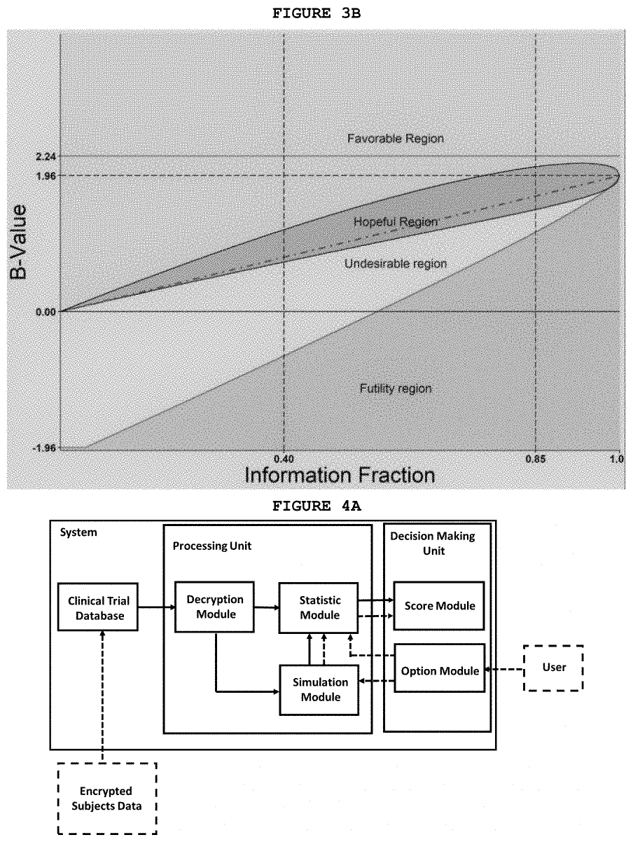

In one embodiment, the present invention provides a radar system with four regions as a monitoring interface for monitoring and guiding an on-going trial. As shown in FIGS. 3A and 3B, the four regions are favorable, hopeful (promising), unfavorable (undesirable), and futility regions.

### The Favorable Region

For simplicity, let us focus on the fixed design for a moment, i.e. C_{α}=Z_{α}. The discussion can be easily extended to the group sequential design. When monitoring an on-going trial, we first want to assess whether the CP under the current “snap-shot” is greater than 1−β (say 90%). In other word, whether B(t)≥b₁(t,1−β)=(Z^{β}\sqrt{1−t}+C_{α})t, derived from Eq. (1)

$$with \hat{θ} = \frac{B ( t )}{t}$$

plugged in for θ, i.e.,

$$C P ( \hat{θ} , t ) = 1 - Φ ( \frac{{C}_{α} - \frac{B ( t )}{t}}{\sqrt{1 - t}} ) = Φ ( \frac{\hat{θ} - {C}_{α}}{\sqrt{1 - t}} ) . \qquad ( 2 )$$

The region {B(t)≥b₁(t, 1−β)} is considered “favorable” as classified by Mehta and Pocock (2011) [25]. b₁(t, 1−β) is the borderline between the favorable and the hopeful regions. In this example (FIGS. 3A and 3B, α=0.025), a selected discrete boundary for rejection region could also be included (depending on the protocol plan) with the O'Brien-Fleming (OBF)—type continuous monitoring boundary (B(t)=2.24), which is placed on the top for an extreme rejection region.

### The Hopeful and Undesirable Regions

Mathematically, the domain of the Brownian motion B (t) may be beyond 1. Let N₀ be the original sample size per arm to meet an unconditional power requirement, 1−β′. The adaptive procedure allows changing the SS at any time, say, at t=n/N₀ with observed B(t). Suppose the new per-arm sample size is N₁>N₀, which corresponds to the information time T₁=N₁/N₀. Let B(T₁) be the potential observation at T₁. To preserve the type-I error rate, the critical boundary C_{α}=C₀ must be adjusted to C₁, so that P(B(T₁)≥C₁|B(t))=P(B(1)≥C_{α}|B(t)) under the null hypothesis. The independent increment property of Brownian motions gives

$$Φ ( \frac{B ( t ) - {C}_{α}}{\sqrt{1 - t}} ) = Φ ( \frac{B ( {T}_{1} ) - {C}_{1} \sqrt{{T}_{1}}}{\sqrt{{T}_{1} - t}} ) .$$

Solving for C₁ produces the following formula for the new critical value:

$${C}_{1} = \frac{1}{\sqrt{{T}_{1}}} \{ \frac{\sqrt{{T}_{1} - t}}{\sqrt{1 - t}} ( {C}_{α} - B ( t ) ) + B ( t ) \} \qquad ( 3 )$$

The same idea used for deriving (1) and (2) produces extended CP given B(t):

$$P ( B ( {T}_{1} ) \geq {C}_{1} \sqrt{{T}_{1}} \vert B ( t ) , \hat{θ} ) = Φ ( \frac{B ( t ) + \hat{θ} ( {T}_{1} - t ) - {C}_{1} \sqrt{{T}_{1}}}{\sqrt{{T}_{1} - t}} ) = Φ ( \frac{\hat{θ} {T}_{1} - {C}_{1} \sqrt{{T}_{1}}}{\sqrt{{T}_{1} - t}} )$$

Setting it to be 1−β and plugging in C₁ from equation (3), we get

$${T}_{1} - t = \frac{{( {Z}_{β} \sqrt{1 - t} + {C}_{α} - B ( t ) )}^{2}}{{\hat{θ}}^{2} ( 1 - t )} \qquad ( 4 )$$

T₁=N₁/N₀ is the new sample size ratio to meet the conditional power 1−β (generally, we may set 1−β=1−β′).

While designing a trial, we may want to control the sample size ratio not to exceed a maximum affordable budget. Let Rₘₐₓ be the maximum sample size ratio to be considered. From Eq. (4), the sample size ratio R for a given desired CP is expressed by

$$R = {T}_{1} = \frac{{N}_{1}}{{N}_{0}} = t + \frac{{( {Z}_{β} \sqrt{1 - t} + {C}_{α} - \hat{θ} t )}^{2}}{{\hat{θ}}^{2} ( 1 - t )} = t + \frac{{( {Z}_{β} \sqrt{1 - t} + {C}_{α} - B ( t ) )}^{2}}{{( B ( t ) / t )}^{2} ( 1 - t )} .$$

Solving for B(t) in terms of a given Rₘₐₓ leads to following inequality,

$$\frac{( {Z}_{β} \sqrt{1 - t} + {C}_{α} ) t}{t + \sqrt{( {R}_{max} - t ) ( 1 - t )}} \leq B ( t ) = \frac{( {Z}_{β} \sqrt{1 - t} + {C}_{α} ) t}{t + \sqrt{( R - t ) ( 1 - t )}} \leq ( {Z}_{β} \sqrt{1 - t} + {C}_{α} ) t = {b}_{1} ( t , 1 - β ) . \qquad ( 5 )$$

Denote

$${b}_{2} ( t , {R}_{max} ) = \frac{( {Z}_{β} \sqrt{1 - t} + {C}_{α} ) t}{t + \sqrt{( {R}_{max} - t ) ( 1 - t )}} .$$

The inequality (5) leads to the “hopeful” region fora given Rₘₐₓ in terms of B-value: b₂(t, Rₘₐₓ)≤B(t)≤b₁(t, 1−β). In the “hopeful” region, the maximum sample size ratio is set to be no more than Rₘₐₓ. Note that the CP under the current “snap-shot” is

$$C P = Φ ( \frac{B ( t ) / t - {C}_{α}}{\sqrt{1 - t}} ) .$$

By replacing B(t)/t with

$$\frac{( {Z}_{β} \sqrt{1 - t} + {C}_{α} )}{t + \sqrt{( {R}_{max} - t ) ( 1 - t )}} ,$$

we map the conditional power in the “hopeful” region so that

$$γ ( t , {R}_{max} ) = Φ ( \frac{{Z}_{β} + {C}_{α} ( \sqrt{1 - t} - \sqrt{{R}_{max} - t} )}{t + \sqrt{( {R}_{max} - t ) ( 1 - t )}} ) \leq C P \leq 1 - β . \qquad ( 6 )$$

This gives another expression of the “hopeful” region in terms of CP. Notice that this Rₘₐₓ-defined lower-bound is a decreasing function of t, from to

$$γ ( 0 , {R}_{max} ) = Φ ( \frac{{Z}_{β} + {C}_{α} ( 1 - \sqrt{{R}_{max}} )}{\sqrt{{R}_{max}}} )$$

γ(1, Rₘₐₓ)=Φ(z_{β}−C_{α}\sqrt{Rₘₐₓ−1}). Note that γ(1, Rₘₐₓ) is the worst case scenario in CP when B(t) falls in the “hopeful” region. When monitoring an on-going trial, we may want to choose a time when CP is not too low (such as <20%) for SSR. Thus, γ(t, Rₘₐₓ) can be used to select an interim analysis time or a time interval for considering SSR.

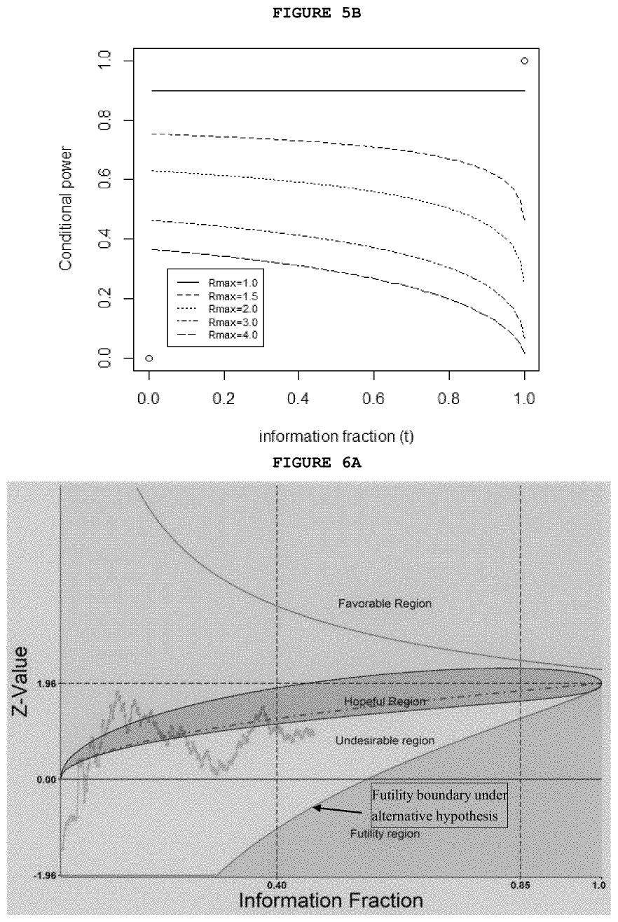

FIGS. 5A and 5B illustrate “favorable” and “hopeful” regions, and the lower bounds of CP, respectively. In FIG. 5B, B(t) falls in the “hopeful” region with the target CP of 1−β=0.90 and Rₘₐₓ=1.5, 2.0, 3.0 and 4.0. In FIG. 5A, the “favorable” region is above the line on top and the “hopeful” regions are between this line and other lines corresponding to different Rₘₐₓ. As shown in FIG. 5A, the larger the Rₘₐₓ is, the lower the borderline of “hopeful” region will be or the larger the “hopeful” region will be. FIG. 5B displays the lower bounds of the CP which also form the corresponding “hopeful” regions in terms of CP. For example, when Rₘₐₓ=2.0, the lower bound of CP ranges from 0.630 (t=0) to 0.248 (t=1).

The region B(t)<b₂ (Rₘₐₓ, t) in FIG. 3 is called “undesirable” region for a moment (only for the sake of being below the “hopeful” region). As the CP on the borderline ranges from 0.630 (t=0) to 0.248 (t=1), it is easy to see that even in the “undesirable” region, we may not want to terminate the trial too early. We need to further define a “futile” region to consider possible early termination.

Futility is also often monitored during a trial, performed either alone or sometimes imbedded with efficacy interim analyses. In either setting, since the decision of whether a trial is futile leading to a decision to stop the trial or not is non-binding, a futility analysis plan should not be used to modify the type-I error rate control. Rather, futility interim analyses increase the type-II error rate, thus induce power loss of the study. What needs to be considered with futility analyses is the power issue. Frequent futility analyses may induce excessive power loss.

How much power loss would be incurred when a trial is continuously monitored for futility? If futility is monitored by a conditional power (CP) (stochastic curtailment) approach, the answer is provided in Lan, Simon and Halperin (1982) [26] as follows. Instead of conditioning on the current estimate \hat{θ}, we use θ*=Z_{α}+Z_{β′} under H_{α}. When the CP (based on θ*) is lower than a threshold (γ_{f}), then the trial is deemed futile and may be stopped for futility. Hence, we construct a continuous futility region in terms of B-value: B(t)≤b_{f}(t)=Φ⁻¹(γ_{f})\sqrt{1−t}−(Z_{α}+Z_{β′})(1−t)+Z_{α}. See the undesirable region in FIG. 3. Compared to the original power 1−β′, the power loss would be at most

$${β}^{′} ( \frac{1}{1 - {γ}_{f}} - 1 ) .$$

For example, if the design power is 0.9 and γ_{f}≤0.5, we can expect the loss to be no more than 0.1. An ending (unconditional) power of 0.8 may be considered acceptable. For γ_{f}=0.20, the power loss is as low as 0.025. The lower the γ_{f} is, the lower the power loss. In general, the power loss with the uniform threshold γ_{f} is negligible.

In practice, occasional futility analyses are performed at pre-specified interim times tᵢ by checking whether CP(tᵢ)<γᵢ, i=1, 2, . . . k. Unlike the continuous boundary where the futility rule is uniformly applied for all t, choosing γᵢ can be flexible depending on the tolerability of CP that we are willing to take at tᵢ. For example, we may choose smaller γᵢ for earlier timepoints compared to later timepoints to avoid early futility stopping. Considering when to conduct futility analyses, we hope the procedure can spot futile situation as soon as possible to save cost as well human suffering from an ineffective therapy. On the other hand, early futility analysis more likely induces power loss for an effective therapy. Thus, we can frame the timing issue of futility analyses as an optimization problem by seeking minimization of the sample size (cost) as the objective while controlling the power loss. This approach which Xi, Gallo and Ohlssen (2017) (27) developed is implemented in DDM.

Note that the “undesirable” region is neither hopeful nor futile. In other word, in this region, owing to the fact that R>Rₘₐₓ, SS increase is infeasible, but the study could not be deemed futile either (CP>γ_{f} under H_{α}). The effect is still in the positive direction (Z- or B-value>0). In this case, we would take no decision/action but continue monitoring.

In summary, let tₖ and dₖ be the k^{th} interim analysis time and stopping boundary, respectively, k=1, 2, . . . K=final. We develop a guidance for monitoring on-going trials in DDM, with the understanding that K is only for planning purpose, not a fixed number.
(1) Stop the trial early for benefit if Z(tₖ)≥dₖ or B(tₖ)≥dₖ\sqrt{tₖ};
(2) If B (tₖ) falls in the “futility” region persistently, we could consider stopping the trial for futility. However, the decision of futility is non-binding.
(3) In between (1) and (2) above, consider SSR or continue monitoring without any change:
   a) No change if CP(tₖ)≥1−β or equivalently B(tₖ)≥(Z_{β}\sqrt{1−tₖ}+C_{α})t, but not exceeds dₖ\sqrt{tₖ}, i.e., in the “favorable” region;
   b) If γ(tₖ,Rₘₐₓ)≤CP(tₖ)<1−β (i.e. B(tₖ) falls in the “hopeful” region), the trial should continue. If we observe consecutive m (say, 10) points in this region, then re-estimate the SS. Choice of Rₘₐₓ depends on sponsor's affordability. For example, set Rₘₐₓ=3 if it is acceptable. When SS is increased, the future boundary values are re-calculated as well.
   c) If B(tₖ) falls in the “undesirable” region, we would take no decision/action but continue monitoring. If B (t) stays in this region persistently, the trial may be recommended for termination for administrative reason such as exceeding the affordable budget.

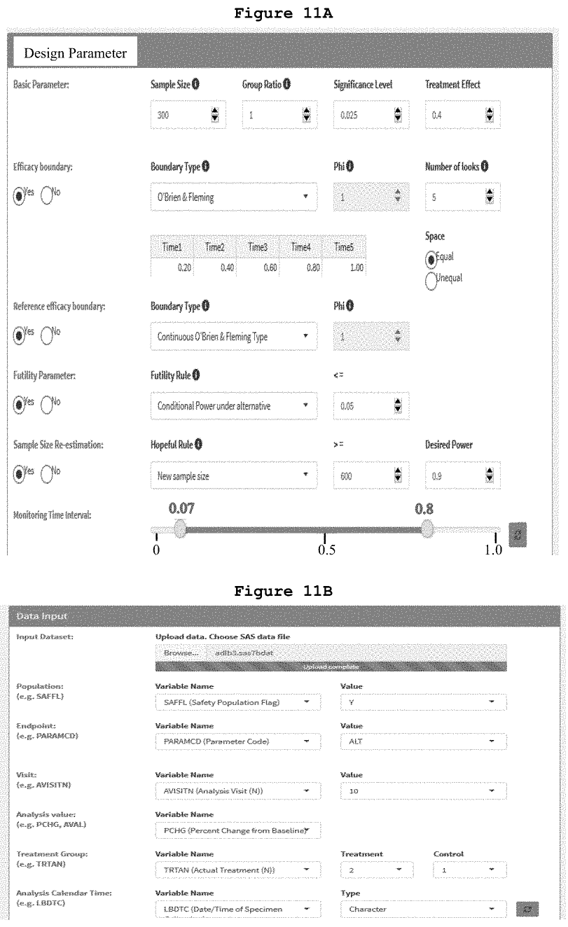

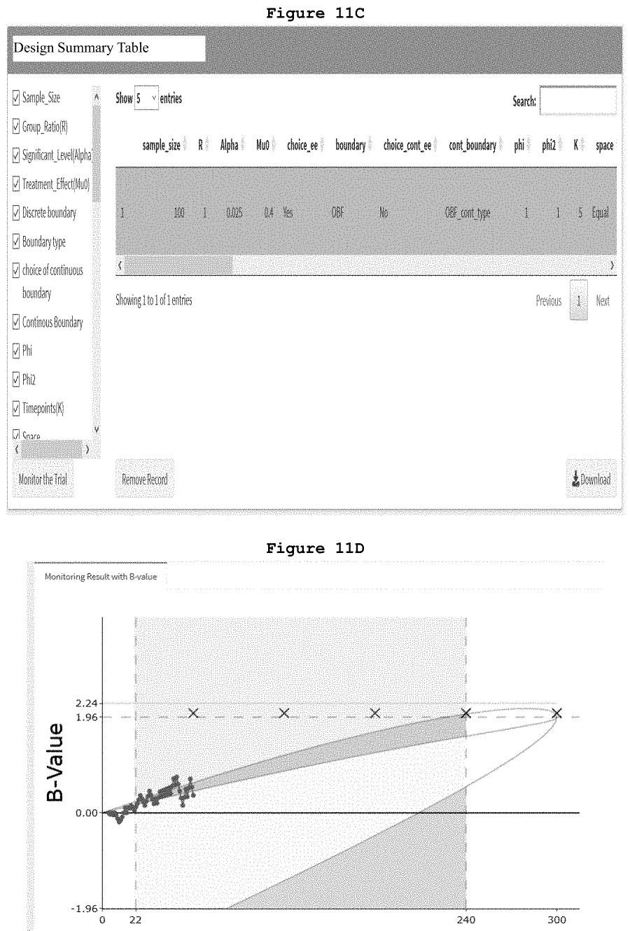

In one aspect, the present invention provides a radar system to dynamically monitor the clinical trial and adapt the boundaries as it proceeds. In one embodiment, the radar system adjusts the region boundaries by adjusting boundary parameters and/or clinical trial parameters. In one embodiment, the present invention provides a graphical user interface (GUI) to monitor the clinical trial based on adjustable boundaries. As a typical example, FIG. 11A illustrates a GUI with parameters for monitoring, FIG. 11B refers to an interface for connection with database and data collection, FIG. 11C is a summary table listing all parameters corresponding to boundaries being monitored in FIG. 11D with three primary regions. FIG. 11D also shows a plot based on data as accumulated. In one embodiment, the boundary parameters include but not limited to CP, the B value, the Z value, the type I or type II error.

In one embodiment, the boundary parameters are set as to align with the goal at particular stage. For example, the ratio (R) of the new sample size to No can be continuously calculated and used to indicate a new sample size to achieve a desired confidential power (CP), such as 95%. In one embodiment, R may be closely monitored as not to exceed a maximum affordable budget (e.g., maximum sample size ratio (Rₘₐₓ) corresponding to the maximum affordable budget). In one embodiment, Rₘₐₓ depends on the phase it falls in and the desired value of the statistical indication (e.g., CP). In one embodiment, the desired CP may be a fixed value, as shown in table 2-1. When t is less than 0.2, Rₘₐₓ can be up to 10 so as to not miss any opportunity due to insufficient data; while when the clinical trial is about to complete, i.e., Rₘₐₓ can be only up to 1.5. In one embodiment, the desired CP may be phase-specific, as illustrated in Table 2-2. For example, at the beginning (t<0.2), a desired CP may be as low as 20% and Rₘₐₓ can be as high as 15. However, when 0.9<t<1.0, since most of data are completed, Rₘₐₓ can be only up to 1.2 to achieve a desired CP of 90%. In one embodiment, a clinical trial can be divided into 2 to 10 stages.

TABLE 2-1
Dependence of Rₘₐₓ on time with a fixed CP

| | t <0.2 | 0.2< t <0.4 | 0.4< t <0.6 | 0.6< t <0.8 | 0.8< t <0.9 | 0.9< t <1.0 |
|---|---|---|---|---|---|---|
| Rₘₐₓ | 10 | 7.5 | 5.5 | 3.0 | 2.0 | 1.5 |

TABLE 2-2
Dependence of Rₘₐₓ on time and phase-specific CP

| | t <0.2 | 0.2< t <0.4 | 0.4< t <0.6 | 0.6< t <0.8 | 0.8< t <0.9 | 0.9< t <1.0 |
|---|---|---|---|---|---|---|
| Rₘₐₓ | 15 | 10 | 7.0 | 3.0 | 1.5 | 1.2 |

In one embodiment, the phase-specific CP is dependent on the existing CP as accumulated. In one embodiment, data trend is also considered in estimating phase-specific CP.

In one embodiment, the phase-specific boundary parameters are provided to the system by a user through an input unit. In one embodiment, the input unit, by operation with a conversion interface or a graphical user interface, transforms a new set of boundary parameters defining new boundaries, or the input from user into a set of signals recognizable by the boundary adjustment module, which translates the signals to a new set of boundary parameters executable by the boundary determination module. In one embodiment, a program integrated as part of the system, e.g., a computerized interface programed with phase-specific CP, upon request, updates the phase-specific boundary parameters.

In one embodiment, the present invention provides a radar system for monitoring on-going trials in DDM. In one embodiment, the radar system categorizes the whole picture into three regions, i.e., an undesirable region, a hopeful region and a favorable region. In one embodiment, the undesirable region comprises a futile region. In one embodiment, the favorable region comprises a successful region. In one embodiment, the system as disclosed by the present invention further provides a recommendation based on the region into which the clinical trial falls. In one embodiment, the boundaries are determined by either Z value or B value.

As shown in FIG. 4F, upon an update or collection of new clinical trial data, the DDM engine (the radar system) evaluates the clinical trial as accumulated, and step 1 determines whether the clinical trial falls into the success region or futility region. If yes, a recommendation of early termination for either success or futility should be provided. Otherwise, i.e., it does not fall into either region, step 2 determines how to proceed further. If it falls into a favorable region, the clinical trial may continue without any modification; if it falls into a hopeful region, the clinical trial may continue with a clinical trial parameter adjustment such as SSR; while if it falls into an undesirable region and if step 3 determines that there is a chance to upgrade to a better region with an affordable SS, the clinical trial may continue with caution. If step 3 determines no chance to upgrade to a better region with an affordable SS, the clinical trial may be terminated for administrative causes. In one embodiment, the present invention provides a method of monitoring clinical trial using the radar system. In one embodiment, the DDM engine is in operation with a Dynamic Trial Design (DTD) which is used for initial clinical trial design based on assumptions. For example, DTD can estimate initial SS based on a) desired values of significance level and power, and b) assumed values of some parameters such as treatment effect. In one embodiment, the DDM engine is in operation with a simulation engine which conducts simulations based on data as accumulated and predicts the future trend and trajectory of the clinical trial.

Assume that a trial is designed with two arms in 1:1 ratio, experimental therapy vs. a standard therapy. The treatment effect is assumed as 0.4, design power 1−β=0.9 and α=0.025 (one-sided). Thus, the initial SS per-arm is N=132. The cap of the SS per-arm (N_{cap}) is set as 600 (i.e. Rₘₐₓ=4.5) and start the monitoring at t=0.4. The desired CP is set as 0.9. Thus, the favorable and hopeful regions are constructed by the borderlines b₁(t)=(1.28\sqrt{1−t}+1.96)t and

$${b}_{2} ( t , {R}_{max} ) = \frac{( 1.28 \sqrt{1 - t} + 1.96 ) t}{t + \sqrt{( {R}_{max} - t ) ( 1 - t )}} .$$

For futility, the continuous futility boundary b_{f}(t)=ϕ(γ_{f})\sqrt{1−t}−θ*(1−t)+1.96 is constructed with scenarios of γ_{f}=0.05, 0.10, 0.15 and 0.20. θ*=δ*\sqrt{T₀}=0.4\sqrt{132/2}=3.25 (=Z_{β}+Z_{α}). The OB-F type boundary is also employed for early efficacy stopping with 5 looks (4 interims and one final) equally spaced (t=0.2, 0.4, 0.6, 0.8, 1). After t=0.4 here for only monitoring SS ratio (R) and futility (0.4, 0.55, 0.70, 0.85). The following procedures are adapted in the simulation.
1) Adjusting SS will be performed only once if the accumulative data (e.g., B-value) fall in the hopeful region for m (e.g., 10) consecutive points and the new final critical boundary will be calculated according to Eq. (3);
2) Futility will be claimed if B(t) below ϕ⁻¹(γ_{f})\sqrt{1−t}−(Z_{β′}+Z_{α})(1−t)+Z_{α}.

TABLE 3
Simulations with the radar system with dynamic and adaptive features

| | | Futility | Rejection | Average | SSR | Futility | Efficacy |
|---|---|---|---|---|---|---|---|
| δₜᵣᵤₑ | γ_{f} | rate | rate | SS | timepoint | timepoint | timepoint |
| 0 | 0.05 | 0.887 | 0.022 | 213 | 0.955 | 0.681 | 0.998 |
| | 0.10 | 0.888 | 0.022 | 202 | 0.955 | 0.639 | 0.998 |
| | 0.15 | 0.889 | 0.022 | 194 | 0.956 | 0.610 | 0.998 |
| | 0.20 | 0.891 | 0.021 | 187 | 0.956 | 0.585 | 0.998 |
| 0.25 | 0.05 | 0.304 | 0.651 | 365 | 0.813 | 0.935 | 0.931 |
| | 0.10 | 0.311 | 0.649 | 362 | 0.812 | 0.919 | 0.931 |
| | 0.15 | 0.318 | 0.643 | 357 | 0.812 | 0.904 | 0.931 |
| | 0.20 | 0.326 | 0.636 | 351 | 0.814 | 0.890 | 0.930 |
| 0.4 | 0.05 | 0.052 | 0.943 | 309 | 0.844 | 0.992 | 0.788 |
| | 0.10 | 0.056 | 0.940 | 307 | 0.843 | 0.988 | 0.787 |
| | 0.15 | 0.061 | 0.937 | 305 | 0.843 | 0.984 | 0.787 |
| | 0.20 | 0.065 | 0.933 | 302 | 0.847 | 0.981 | 0.786 |

Note:
# of simulation = 100,000 and monitoring started at t = 0.4.
If SSR or futility stopping or efficacy stopping were not performed, the respective timepoint was set to 1.

As can be seen through the simulations (Table 3):
1) Type I error rate is well controlled;
2) When δₜᵣᵤₑ=0, the futility is detected at a relatively early stage (0.59-0.68) with detection rate>85%;
3) When δₜᵣᵤₑ>0, the actual powers are slightly greater than the target power corresponding to N=132 per-arm due to the built-in SSR showing no power-loss;
4) When treatment effect is over-assumed (δₜᵣᵤₑ=0.25), the SSR is performed around t=0.81 on average. When treatment effect is assumed correctly (δₜᵣᵤₑ=0.40), early efficacy is claimed around t=0.79.

The above simulations demonstrate that the radar system with dynamic and adaptive features works well in the given settings.

### Use of Radar System by DMC

In most Phase II-III clinical trials, Data Monitoring Committee (DMC) periodically monitors the safety and/or efficacy and usually meets every 3 to 6 months depending on the disease and specific intervention. For example, DMC may meet more frequently at early stage to understand the safety profile sooner, or meet more frequently for an oncology trial with new regimen in comparison to a trial for non-life threating disease. The current practice for DMC involves three parties: Sponsor, Independent Statistical Group (ISG) and DMC. The sponsor is to conduct and manage the on-going study. The ISG prepares blinded and unblinded data packages: tables, listing and figures (TLFs) based on scheduled data cut (usually more than a month before the DMC meeting). The preparation work is usually time-consuming, which takes about 3-6 months. The traditional DMC practice has some disadvantages. First, the data package for each interim analysis only reflects a snapshot of the data and does not show the trend of treatment effect (efficacy or safety). Second, unblinding data and preparation of data package are time-consuming. Usually, it takes about 3-6 months for ISG to unblind data and prepare data package for DMC's review. Human involvement may introduce errors.

In another important aspect, the radar system of the present invention is applied to trials on urgent need under a pandemic crisis, such as the COVID-19. Monitoring the outcome (e.g., safety and efficacy) and adjusting the clinical trial in a nearly continuous and timely fashion are highly needed and challenging. The conventional way would cost a lot of lives and budget because of its low efficiency and inflexibility as discussed above. In one embodiment, the radar system with the dynamic and adaptive features can collect, unblind, and analyze the data on a real-time basis, and provide suggestion as to how to manage or adjust the clinical trial in view of the data as accumulated in a timely manner.

That degree of availability is necessary for the data and safety monitoring committee (DSMC) to perform its role effectively. In 10^{th} annual conference at the University of Pennsylvania held in 2018 [28], Dr. Janet Wittes said that all the data, not just specific variables, must be available to the independent statistician all the time, not only just before a meeting. The radar system as well as the detection methods in this invention can be directly applied to DSMC. Such application does not affect the execution of the clinical trial nor the independence of data monitoring or analysis. By integrating with the EDC/IWRS system, the radar system can create a seamless data monitoring ecosystem. In one embodiment, the present invention can construct a trial radar system using the pre-specified parameters (e.g. efficacy and/or futility boundaries) and the status regions (as discussed above). In one embodiment, the present invention can construct regions/boundaries using the then-specified parameters (e.g. efficacy and/or futility boundaries) and the status regions (as discussed above). In one embodiment, the then-specified parameters are determined in view of the then-available clinical trial data and guidance, e.g., maximum budget. In one embodiment, the accumulative trial data of interest (e.g., efficacy and safety) can be displayed via a display module or a graphical user interface in connection with the radar system. In one embodiment, the radar system suggests not only just the go/no go at the interim analysis, but also provide guidance on a real-time basis to reach its final destination. In one embodiment, to minimize potential operational bias, the radar system allows data access with authorization via an authorization module. In one embodiment, the radar system is accessible only by DSMC members with encryption. In one embodiment, the radar system only presents the results at specified time, e.g., DSMC meeting. In one embodiment, for the purpose of closely monitoring the drug safety, DSMC may require turning on the only safety portion display so that it can be monitored directly in real time fashion.

In one embodiment, the radar system of the present invention may be used in the following applications:
Trial Diagnosis. The radar system can be retrospectively applied to completed studies to learn what was going on during the trial and the key factors that cause the outcomes. This can be applicable for all types of studies, including these failed ones. See Examples.
Drug safety detection. The radar system can continuously monitor safety of drug or candidate and detect signal.
Dose selection. The radar system can be used for a seamless, optimal phase 2/3 combination trial by identifying most potential doses for phase 3.
Population selection. The radar system can identify the subpopulation in which the drug is most effective and be directly applied to RCT or RWE setting for personalized medicine.

In one embodiment, the present invention provides a graphical user interface-based system for monitoring and guiding an ongoing clinical trial on an adjustable and real-time basis, comprising:
a. a clinical trial database for storing information from an ongoing clinical trial, wherein said information comprises a set of subject data that is being continuously updated as said ongoing clinical trial proceeds;
b. a boundary determination module for determining boundaries for a group of regions comprising a favorable region, a hopeful region and an undesirable region, wherein said boundaries are subject to boundary adjustment as said ongoing clinical trial proceeds, wherein each region represents a different level of risk associated with an accumulative effect of said ongoing clinical trial; and
c. a graphical user interface (GUI), operable with said boundary determination module, for displaying a plot of said accumulative effect of said ongoing clinical trial and boundary parameters corresponding to said group of regions, wherein said GUI allows a user to adjust values of boundary parameters in view of said plot, thus generating new boundaries on a real-time basis as said ongoing clinical trial proceeds, wherein said accumulative effect of said ongoing clinical trial is continuously projected onto said plot, thereby monitoring and guiding said ongoing clinical trial on an adjustable and real-time basis.

In one embodiment, the set of subject data comprises unblinded data or one or more accumulative effects derived from said unblinded data.

In one embodiment, the undesirable region comprises a futility region, and said favorable region comprises a successful region.

In one embodiment, the GUI provides a recommendation depending on the region into which said ongoing clinical trial falls, wherein said recommendation is:
a. “early termination for success” if said accumulative effect falls into said successful region;
b. “early termination for futility” if said accumulative effect falls into said futility region;
c. “continuation without modification” if said accumulative effect falls into said favorable region but not said successful region;
d. “continuation with sample size re-estimation” if said accumulative effect falls into said hopeful region; or
e. “continuation with caution” if said accumulative effect falls into said undesirable region but not futility region.

In one embodiment, the accumulative effect is one or more statistical scores selected from the group consisting of Score statistics (B value), Wald statistics (Z value), point estimate B, and 95% confidence interval, conditional power (CP), type I error and type II error.

In one embodiment, the boundary parameters have desirable values that are phase- or time-specific.

In one embodiment, the system is in operation with a simulation module which conducts simulations in view said set of subject data as accumulated and its trend of said plot, predicts trend and trajectory of said ongoing clinical trial in the future and optionally proposes a clinical trial parameter adjustment by comparing with an initial or existing clinical trial design and assumptions used for said initial or existing clinical design.

In one embodiment, the simulations are conducted with a trend analysis.

In one embodiment, the trend analysis is a piecewise linear analysis in which different weights are assigned to each piece showing a linear trend.

In one embodiment, the the favorable region corresponds to a region where the B value is no less than b₁(t, 1−β); the hopeful region corresponds to a region where the B value is no more than b₁(t, 1−β) but no less than b₂(t, Rₘₐₓ); and the undesirable region corresponds to a region wherein the B value is less than b₂(t, Rₘₐₓ); wherein said Rₘₐₓ is a maximum sample size ratio of said ongoing clinical trial at time t.

In one embodiment, the futility region corresponds to a region wherein the B value is no more than b_{f}(t), wherein b_{f}(t) is the threshold value at time t indicating a statistically significant conclusion for futility and said successful region corresponds to a region wherein the B value is no less than Cs, wherein Cs is the threshold value indicating a statistically significant conclusion for success.

In one embodiment, the group of regions in said plot are marked by different colors or patterns.

In one embodiment, when said ongoing clinical trial falls into said the hopeful region for 10 points consecutively, the system provides a signal indicating necessity to adjust one or more clinical trial parameters of said ongoing clinical trials.

In one embodiment, the present invention provides a graphical user interface-based method for monitoring and guiding an ongoing clinical trial on an adjustable and real-time basis, comprising:
a. storing information from an ongoing clinical trial into a clinical trial database, wherein said information comprises a set of subject data that is being continuously updated as said ongoing clinical trial proceeds;
b. mapping boundaries, via a boundary determination module, for a group of regions comprising a successful region, a favorable region, a hopeful region, an undesirable region and a futility region, wherein said boundaries are subject to boundary adjustment as said ongoing clinical trial proceeds, wherein each region represents a different level of risk associated with an accumulative effect of said ongoing clinical trial;
c. conducting said boundary adjustment on a graphical user interface (GUI), wherein said GUI displays a plot of said accumulative effect of said ongoing clinical trial and boundary parameters corresponding to said group of regions, said GUI allows a user to adjust values of said boundary parameters in view of said plot, thus generating new boundaries on a real-time basis as said ongoing clinical trial proceeds, wherein said accumulative effect of said ongoing clinical trial is continuously projected onto said plot; and
d. providing, via said GUI, a recommendation guiding said ongoing clinical trial, wherein, depending on which region said ongoing clinical trial falls into, said recommendation is
   1) “early termination for success” if said accumulative effect falls into said successful region;
   2) “early termination for futility” if said accumulative effect falls into said futility region;
   3) “continuation without modification” if said accumulative effect falls into said favorable region but not said successful region;
   4) “continuation with sample size re-estimation” if said accumulative effect falls into said hopeful region; or
   5) “continuation with caution” if said accumulative effect falls into said undesirable region but not futility region

In one embodiment, the present invention provides a graphical user interface-based method for diagnosing an already completed clinical trial, comprising:
a. sequentially applying information from an already completed clinical trial into a clinical trial database according to time of patient data completion, wherein said information comprises a set of subject data that is being continuously updated;
b. mapping boundaries, via a boundary determination module, for a group of regions comprising a successful region, a favorable region, a hopeful region, an undesirable region and a futility region subject to boundary adjustment as said information is being applied, wherein each region represents a different level of risk associated with an accumulative effect of said ongoing clinical trial;
c. conducting said boundary adjustment on a graphical user interface (GUI), wherein said GUI displays a plot of said accumulative effect of said ongoing clinical trial and boundary parameters corresponding to said group of regions, said GUI allows a user to adjust values of said boundary parameters in view of said plot, thus generating new boundaries assuming said clinical trial was proceeding, wherein said accumulative effect of said clinical trial is continuously projected onto said plot; and
d. providing, via said GUI, a diagnosis of said clinical trial, wherein, assuming said clinical trial was proceeding, depending on the region into which said clinical trial falls, said diagnosis is
   1) “early termination for success” if said accumulative effect falls into said successful region;
   2) “early termination for futility” if said accumulative effect falls into said futility region;
   3) “continuation without modification” if said accumulative effect falls into said favorable region but not said successful region;
   4) “continuation with sample size re-estimation” if said accumulative effect falls into said hopeful region; or
   5) “continuation with caution” if said accumulative effect falls into said undesirable region but not futility region

In one embodiment, the present invention provides a radar system for monitoring and guiding an ongoing clinical trial on an adjustable and real-time basis, comprising:
a. a clinical trial database for storing information from an ongoing clinical trial, wherein said information comprises a set of subject data that is being continuously updated as said ongoing clinical trial proceeds;
b. a boundary determination module for determining boundaries for a group of regions comprising a favorable region, a hopeful region and an undesirable region, wherein said boundaries are subject to boundary adjustment as said ongoing clinical trial proceeds, wherein each region represents a different level of risk associated with an accumulative effect of said ongoing clinical trial;
c. an interactive boundary adjustment module, operable with said boundary determination module, for conducting said adjustment adjusting existing boundaries into new boundaries in view of said plot on a real-time basis as said ongoing clinical trial proceeds; and
d. a display module for continuously projecting said accumulative effect of said ongoing clinical trial onto a plot comprising said group of regions, thereby monitoring and guiding said ongoing clinical trial on an adjustable and real-time basis.

In one embodiment, the set of subject data comprises unblinded data or one or more accumulative effects derived from said unblinded data.

In one embodiment, the undesirable region comprises a futility region, and said favorable region comprises a successful region.

In one embodiment, the GUI provides a recommendation depending on which region said ongoing clinical trial falls into, wherein said recommendation is:
1) “early termination for success” if said accumulative effect falls into said successful region;
2) “early termination for futility” if said accumulative effect falls into said futility region;
3) “continuation without modification” if said accumulative effect falls into said favorable region but not said successful region;
4) “continuation with sample size re-estimation” if said accumulative effect falls into said hopeful region; or
5) “continuation with caution” if said accumulative effect falls into said undesirable region but not futility region.

In one embodiment, the accumulative effect is one or more statistical scores selected from the group consisting of Score statistics (B value), Wald statistics (Z value), point estimate \hat{θ}, and 95% confidence interval, conditional power (CP), type I error and type II error.

In one embodiment, the boundary adjustment module adjusts, in view of said plot, existing boundaries to new boundaries by translating a new guidance into a new set of boundary parameters defining said new boundaries.

In one embodiment, the new set of boundary parameters reflect desirable values that are phase- or time-specific.

In one embodiment, the radar system is in operation with a simulation module which conducts simulations in view of said set of subject data as accumulated and its trend of said plot, predicts trend and trajectory of said ongoing clinical trial in the future and optionally proposes a clinical trial parameter adjustment by comparing with an initial or existing clinical trial design and assumptions used for said initial or existing clinical design.

In one embodiment, the simulations are conducted with a trend analysis.

In one embodiment, the trend analysis is a piecewise linear analysis in which different weights are assigned to each piece showing a linear trend.

In one embodiment, the favorable region corresponds to a region where the B value is no less than b₁(t, 1−β); the hopeful region corresponds to a region where the B value is less than b₁(t, 1−β) but no less than b₂(t, Rₘₐₓ); and the undesirable region corresponds to a region wherein the B value is less than b₂(t, Rₘₐₓ); wherein said Rₘₐₓ is a maximum sample size ratio of said ongoing clinical trial at time t.

In one embodiment, the futility region corresponds to a region wherein the B value is no more than b_{f}(t), wherein b_{f}(t) is the threshold value at time t indicating a statistically significant conclusion for futility and said successful region corresponds to a region wherein the B value is no less than Cs, wherein Cs is the threshold value indicating a statistically significant conclusion for success.

In one embodiment, the group of regions in said plot are marked by different colours or patterns.

In one embodiment, when said ongoing clinical trial falls into said the hopeful region for 10 points consecutively, the radar system provides a signal indicating necessity to adjust one or more clinical trial parameters of said ongoing clinical trials.

In one embodiment, the present invention provides a method for monitoring and guiding an ongoing clinical trial on an adjustable and real-time basis, comprising:
a. storing information from an ongoing clinical trial into a clinical trial database, wherein said information comprises a set of subject data that is being continuously updated as said ongoing clinical trial proceeds;
b. mapping boundaries, via a boundary determination module, for a group of regions comprising a successful region, a favorable region, a hopeful region, an undesirable region and a futility region, wherein said boundaries are subject to boundary adjustment as said ongoing clinical trial proceeds, wherein each region represents a different level of risk associated an accumulative effect of said ongoing clinical trial;
c. conducting said boundary adjustment, via an interactive boundary adjustment module, adjusting values of said boundary parameters in view of said plot, thus generating new boundaries on a real-time basis as said ongoing clinical trial proceeds;
d. continuously projecting, via a display module, said accumulative effect of said ongoing clinical trial onto a plot comprising said group of regions; and
e. providing, via said display module, a recommendation guiding said ongoing clinical trial, wherein, depending on which region said ongoing clinical trial falls into, said recommendation is:
   1) “early termination for success” if said accumulative effect falls into said successful region;
   2) “early termination for futility” if said accumulative effect falls into said futility region;
   3) “continuation without modification” if said accumulative effect falls into said favorable region but not said successful region;
   4) “continuation with sample size re-estimation” if said accumulative effect falls into said hopeful region; or
   5) “continuation with caution” if said accumulative effect falls into said undesirable region but not futility region.

### Examples

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1 Application of the Radar System to the First Clinical Trial of Remdesivir in Adult Patients with Severe COVID-19

The first double-blind, placebo-controlled clinical trial on the potential antivirus effect of Remdesivir in adult patients with severe COVID-19 was conducted in Wuhan, China (Wang et al., 2020) [29] during January to March, 2020. The trial was globally watched during the pandemic crisis and the trial's DMC was commissioned to make quick and scientifically sound decisions. The DMC faced real challenge to be highly efficient in data transmission and monitoring on key efficacy and safety data, and to function in a very timely manner. The DMC decided to use the eDMC™ software (CIMS Global) with our DDM “trial-radar” to monitor on-going key safety and efficacy data almost weekly as patients enrolled quickly (Shih, Yao & Xie, 2020) [30]. The key efficacy endpoints planned for DMC to monitor was the 6-point ordinal score of the clinical conditions of patients on Days 7, 14, 21, and 28. (However, at early review meetings, DMC also requested instant looks at data for Days 3, 5 and 10, which were considered exploratory.)

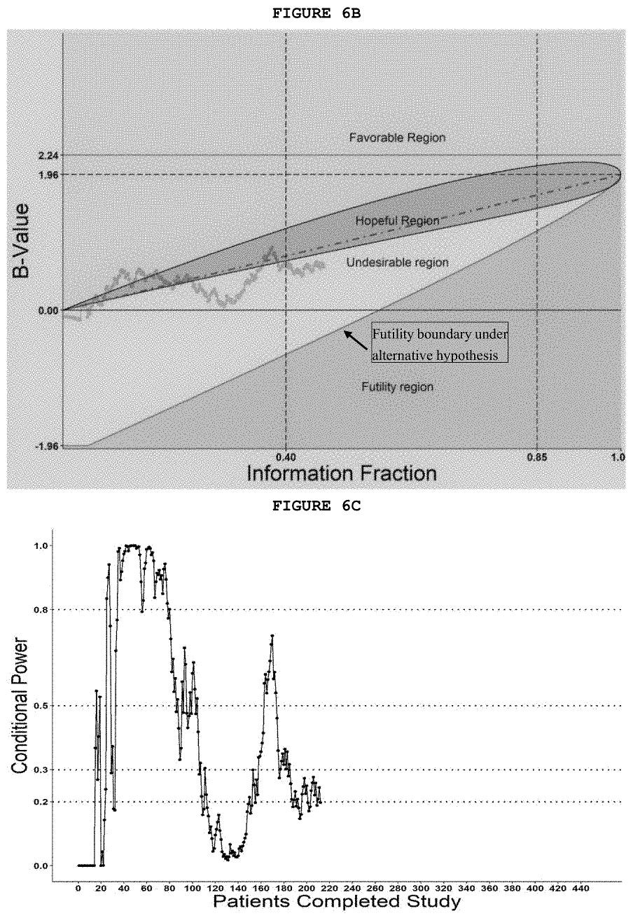

According to the plan of DMC's charter, the treatment groups were compared with respect to their distributions of the ordinal scale using the stratified Wilcoxon-Mann-Whitley (WMW) Rank-sum test. As the trial progressed, the trend of the tests was monitored as patients accumulate and treatment days expand. The distribution data were displayed by bar charts and the WMW Rank-sum tests were followed on the DDM “radar” screen. The “radar” screen was constructed with regions of CP to show whether the Rank-sum test was in “favorable”, “hopeful”, “undesirable” or “futile” regions. The trace of the Rank-sum test signals the trend of the trial result from time-to-time as patients being enrolled. During the early stage of the trial, more data cumulated in earlier days and fewer data in later days of follow-up, as expected. Thus, the data examinations were exploratory. Only if a consistent strong signal indicated by the rank-sum test (i.e., falling in the favorable region), would the formal analysis on the protocol-designed primary endpoint, time to clinical improvement (TTCI), be triggered. As time progressed, more patients had longer follow-up data, as expected. Most of times, exploratory analysis was performed, as planned, by examining the “radar” graphs. However, in case it was needed to protect against an inflated false positive rate, especially at the later stage of the trial when sufficient number of patients were enrolled/followed up and we would examine multiple Rank-sum tests on Days 7, 14, 21 and 28, the DMC planned to use Hochberg's step-wise procedure for protecting an overall alpha at 0.025 (1-sided, or 0.05 2-sided) level for this ordinal (secondary) endpoint. Since there was no idea when and how many times the TTCI analysis would be triggered, the group sequential flexible alpha-spending function approach was designed to maintain the overall alpha of 0.025 (1-sided, or 0.05 2-sided) level for the TTCI primary endpoint as well. Moreover, in anticipation of the fast-pace enrollment and relatively short trial duration, and in consideration of the urgent matter for the study, the DMC chose the Pocock-type alpha-spending function for this primary endpoint. Note that the Pocock-type alpha-spending function being concave rather than convex, indicating that more alpha would be spent at earlier than later time, fits the urgent situation of the epidemic; see Shih, Yao & Xie (2020) [30]. Here, FIGS. 6A and 6B demonstrate the path of the Day 28 WMW Rank-sum test Z-values and B-values on the DDM “radar” screen near the fifth DMC meeting around the end of March 2020 when 212 patients (out of 453 planned) finished the Day-28 study treatment and evaluation. The hopeful region was set to have CP satisfying Eq. (6) with Rₘₐₓ=3, and 1−β=0.90. The dash-dot boundary line inside represents CP=50%.

As shown, at early DMC meetings when less than 100 patients (t=0.22) had their Day-28 evaluations, data fluctuated across favorable and hopeful regions, but the CP was greater than 50% most of the times after about 40 patients (t=0.088). The DMC was optimistic and recommended continuation of the trial. Later, however, the CP fell lower than 50% most of the times, and lingered in the unhopeful region when more patients completed their Day-28 evaluations. At the 4^{th} DMC meeting when the Day-28 data from about 180 patients were evaluated, CP was approximately 33%, thus an increase of the SS was considered. However, the sponsor informed the DMC that the pandemic was already brought under control in China and the study could not continue even with the original SS, let alone an increase. The study bailed out due to incomplete enrollment on Apr. 2, 2020. The usefulness of the DDM was well demonstrated in this trial.

A weighted average trend was also performed using 4-piecewise linear drift in the B-value plot using the following 4 segments when 40, 140, 170, and 212 patients completed, i.e., at t₁=0.088, t₂=0.309, t₃=0.375, and t₄=0.468, respectively. In the B-value plot, the slopes were

$${S}_{1} = \frac{B ( 0.088 )}{0.088} = \frac{0.3365}{0.088} = 3.82 ,$$

$${S}_{2} = \frac{B ( 0.309 ) - B ( 0.088 )}{0.309 - 0.088} = \frac{0.1269 - 0.3365}{0.309 - 0.088} = - 0.95 ,$$

$${S}_{3} = \frac{B ( 0.375 ) - B ( 0.309 )}{0.375 - 0.309} = \frac{0.9069 - 0.1269}{0.375 - 0.309} = 11.82 , and$$

$${S}_{4} = \frac{B ( 0.468 ) - B ( 0.375 )}{0.468 - 0.375} = \frac{0.6266 - 0.9069}{0.468 - 0.375} = - 3 \text{.01} ,$$

respectively for these four segments. We choose to down weigh the trend at earlier times, by using w₁=0.05,w₂=0.30, w₃=0.30, w₄=0.35 for a sensitivity analysis in addition to the Brownian motion model's weights ( 40/220=0.18, 100/220=0.45, 30/220=0.14, 50/220=0.23). The resulting weighted average slope was w₁S₁+w₂S₂+w₃S₃+w₄S₄=2.40.

With 2.40 as the estimate of θ in Eq. (1), the conditional power CP(61,0=0.469, compared to the snapshot estimate \hat{θ}=0.6266/0.468=1.34 and CP(\hat{θ}, t)=0.198. This sensitivity analysis would put the CP in the hopeful region based on the path of the data with subjective weighs. A suggestion for increasing of SS would be made (and not be accepted by the sponsor due to the same reason of lack of patients since the pandemic was already under control in this case).

### Example 2 Application of the Radar System for Trial Diagnosis on a Positive Study

This was a multi-center, double-blind, placebo-controlled study with two weeks of daily oral administration of an experimental drug or placebo in subjects with nocturia. The primary endpoint of the study was the 14-day average number of nocturnal voids. The original design was a fixed size design with 80% power at one-side alpha=0.025. Total of 83 subjects were randomized into the study. At the final analysis, the group with the experimental drug was shown significantly superior to the placebo group (Z-test compared to 1.96).

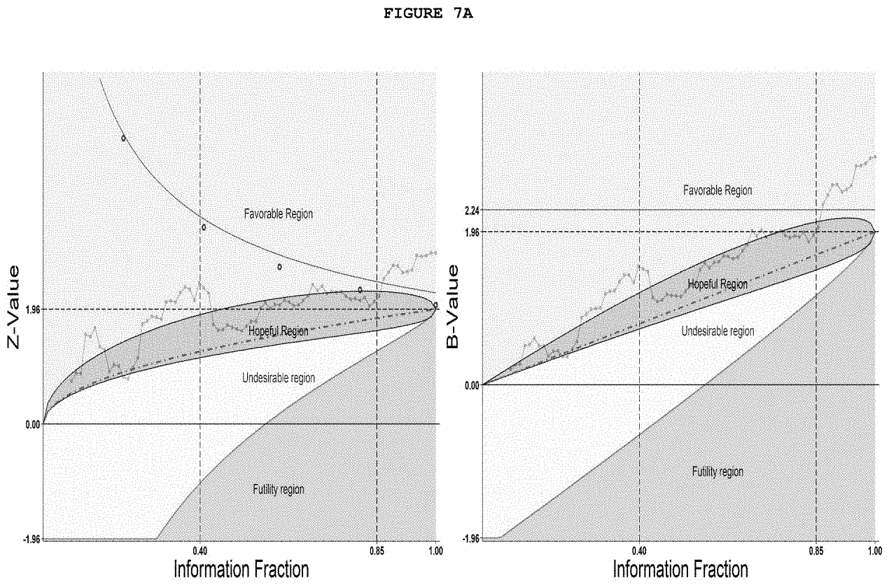

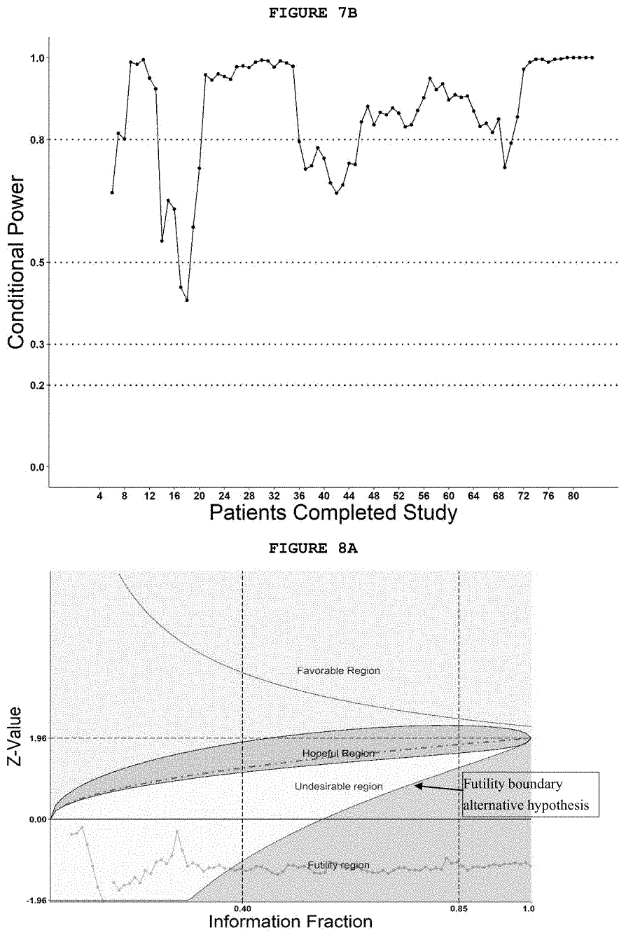

We have reconstructed the study retrospectively to demonstrate what happened if patients were sequentially monitored with the DDM system according to their time of completing the 14 days of treatment. FIGS. 7A and 7B display the DDM radar screen plots and the CP. As seen, whether with the “continuous” or discrete OB-F boundary (equally spaced, five open blue circles), the test did not cross the corresponding boundaries until t>0.85. The potential success of the study can be shown from the CP plot: the CP was above 80% most of the times starting t>0.55, after 46 subjects completed the study. This example also demonstrated (1) fluctuations occur in early part of a trial; (2) SSR should not be considered too early when data are still uncertain; (3) CP>80% during near half of the study, continuing monitoring the trial is helpful; SSR is most likely not needed.

### Example 3 Application of the Radar System for Trial Diagnosis on a Negative Study

This randomized, double-blind, placebo-controlled study assessed the safety and efficacy of an orally administered experimental drug in patients with nonalcoholic fatty liver disease (NAFLD). The primary endpoint was the change in serum ALT (alanine transaminase) from baseline to 6 months. 91 subjects were randomized to 3 active (dose) groups and placebo. The original design was a fixed size design with 80% power at a one-side alpha=0.025. At the final analysis, the active groups were shown to be significantly inferior to the placebo group.

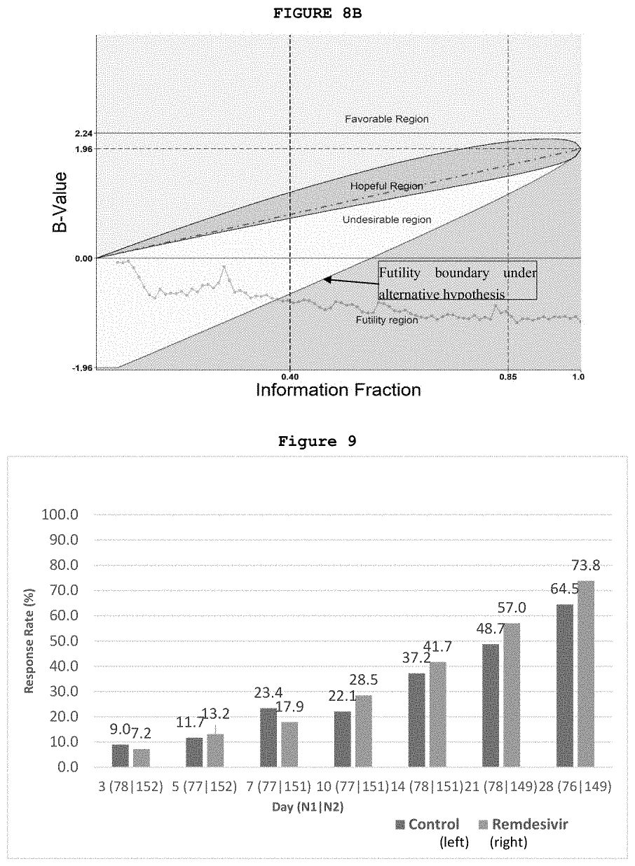

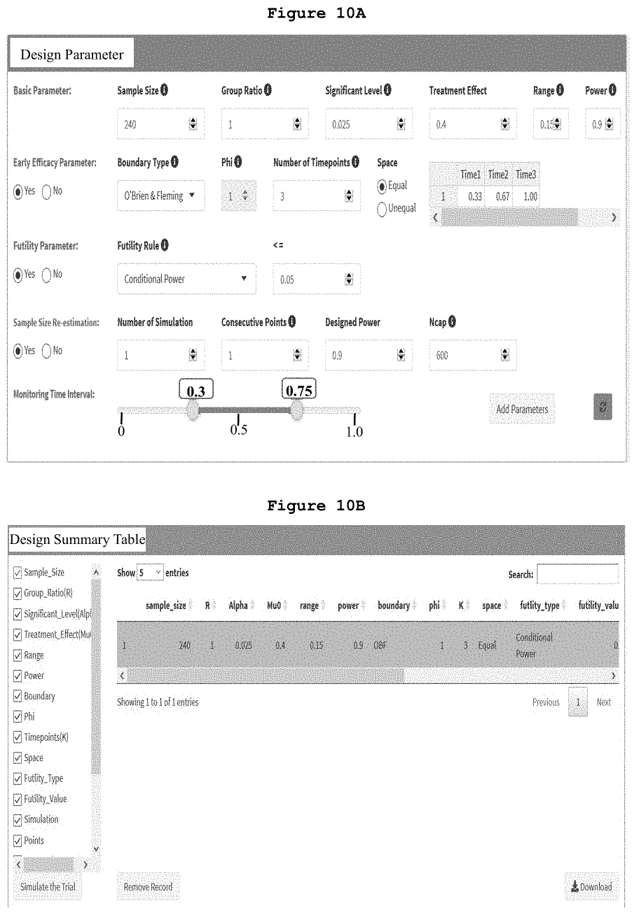

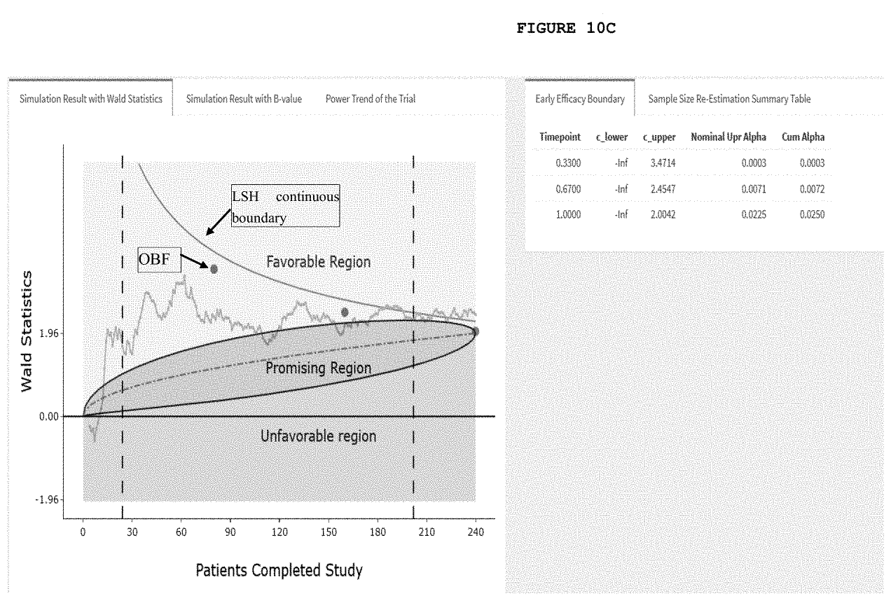

Again, we reconstructed the study retrospectively to demonstrate what happened if patients were sequentially monitored with the DDM system according to their time of completing the 6 months of treatment. FIGS. 8A and 8B display the DDM screen plots of combined active groups vs. placebo and the CP. As seen, the Z-values were below zero and the conditional powers were nearly zero from the beginning of the study until end. The DDM plots showed the trial entered from the unhopeful region to the futile zone after t=0.40. The study could have been early terminated for futility if DDM had been used. One might argue that DDM is hardly needed in this extremely negative case. However, since futility is non-binding, it might also be possible that one time or even more interim analyses with snapshot data would not convince the sponsor to abandon the trial, unless the path of the data showed clearly the hopeless trend, which could be provided by the DDM. As discussed above, the clinical trial in the undesirable region may continue with caution. If the sponsor would really desire another try at t=0.40 even at a risk higher than the one in the current design, it may lower the boundary line so that the clinical trial is in the undesirable region for a moment, justifying that the clinical trial may continue with caution. Once the clinical trial re-travels into the futile region under the new boundary, it may decide how to proceed by then. In one embodiment, the trend of plot is also considered in redefining boundary. As shown in FIG. 8B, because the overall trend between t=0 and t=0.35 is negative, which indicates that there is almost no chance to travel back, the sponsor may increase the boundary line for futility, thus shifting the point at t=0.35 to the futility region, which indicates that the clinical trial shall be terminated at t=0.35.

### Example 4 the Radar System Applied to the First Remdesivir Trial on COVID (Example 1) Triggered a Re-Analysis

The first double-blind, placebo-controlled, randomized trial on intravenous remdesivir for treating severe COVID-19 patients conducted in Wuhan, China [31] was highly watched. The main results [29] received global attention. However, the study stopped early after 237 of the planned 453 patients were enrolled because of insufficient patents. That report messaged that no statistically significant benefits from remdesivir were observed beyond those of standard care. The result was contradicted to the result from a similar trial on remdesivir in US [34] which was first announced by Dr. Fauci on Apr. 29, 2020.

The China trial was monitored using the radar system. By reviewing the treatment effect charts on the radar system over days 5, 10, 14, 21, and 28, it was discovered that the treatment effect of remdesivir had crossed the success stopping boundary at days 10 and 14 indicating a superiority of remdesivir over placebo in treating patients with COVID-19. This discovery triggered a re-analysis on the China data.

Specifically, the report indicated that remdesivir treatment was not associated with a difference in time to clinical improvement (TTCI), expressed by a hazard ratio of 1.23 [95% CI: 0.87-1.75]. The median TTCI was 21 days in the remdesivir group vs 23 days in the control group, for the 28-day trial. The study defined the primary endpoint as two-point reduction in patients' admission status on a 6-point ordinal scale, or live discharge from the hospital, whichever came first. The 6-point scale was 6=death; 5=hospitalization, requiring extracorporeal membrane oxygenation (ECMO) and/or invasive mechanical ventilation (IMV); 4=hospitalization, requiring non-invasive ventilation (NIV) and/or high-flow oxygen therapy (HFNC); 3=hospitalization, requiring supplemental oxygen (but not NIV/HFNC); 2=hospitalization, but not requiring supplemental oxygen; 1=hospital discharge or meets discharge criteria (discharge criteria are defined as clinical recovery, i.e. fever, respiratory rate, oxygen saturation return to normal, and cough relief, all maintained for at least 72 hours); see Table 4. Scale=3 represents moderately severe and scale=4 and 5 represent critically sever categories.

TABLE 4
Scale chart

| Scale | 6 | 5 | 4 | 3 | 2 | 1 |
|---|---|---|---|---|---|---|
| Chinese | Death | Hospitalization, | Hospitalization, | Hospitalization, | Hospitalization, but | Hospital discharge |
| Trial | | requiring | requiring | requiring | not requiring | or meets discharge |
| | | ECMO | NIV and/or | supplemental oxygen | supplemental oxygen | criteria (discharge |
| | | and/or | high-flow | (but not | | criteria are defined |
| | | IMV | oxygen | NIV/HFNC) | | as clinical recovery, |
| | | | therapy | | | i.e. fever, respiratory |
| | | | (HFNC) | | | rate, oxygen |
| | | | | | | saturation return to |
| | | | | | | normal, and cough |
| | | | | | | relief, all maintained |
| | | | | | | for at least 72 hours). |

| Scale | 1 | 2 | 3 | 4 | 5 | 6 | 7 |
|---|---|---|---|---|---|---|---|
| ACTT - | Death | Hospitalized, | Hospitalized, | Hospitalized, | Hospitalized, not | Not | Not |
| version 1 | | on invasive | on non-invasive | requiring | requiring | hospitalized, | hospitalized, |
| | | Mechanical | Ventilation | supplemental oxygen | supplemental oxygen | limitation on | No limitations |
| | | ventilation | or high flow | | | activities | on activities |
| | | or ECMO | Oxygen devices | | | | |

| Scale | 1 | 2 | 3 | 4 | 5 | 6 | 7 | 8 |
|---|---|---|---|---|---|---|---|---|
| ACTT - | Death | Hospitalized, on | Hospitalized, on | Hospitalized, | Hospitalized, not | Hospitalized, | Not hospitalized, | Not hospitalized, |
| version 2 | | invasive | non-invasive | Requiring | Requiring | not | limitation on | no limitations |
| | | mechanical | ventilation | supplemental | supplemental | requiring | activities | on activities |
| | | ventilation | or high flow | oxygen | oxygen - | supplemental | and/or requiring | |
| | | or ECMO | oxygen | | requiring | oxygen - | home oxygen | |
| | | | devices | | ongoing | no longer | | |
| | | | | | Medical care | Requires | | |
| | | | | | (COVID-19 related | Ongoing | | |
| | | | | | or otherwise) | medical care | | |

ECMO: extracorporeal membrane oxygenation;
NIV: non-invasive ventilation;
IMV: invasive mechanical ventilation;
HFNC: High flow nasal cannula

In contrast, the preliminary results of the Adaptive COVID-19 Treatment Trial (ACTT) [34, 35] showed that remdesivir led a 31% faster recovery than the standard care treatment. Specifically, the median time to recovery was 11 days for patients treated with remdesivir compared with 15 days for those who received placebo (p<0.001) [34]. With the high statistical significance, the trial was early stopped and was renamed “ACTT-1”, as remdesivir became the “standard of care” for the rest of the trial as part of the adaptive design [36, 37]. Contrary to the Chinese trial, this preliminary result from interim data suggests a possible “over-power” scenario for ACTT-1.

To mitigate the difference between a seemly “under-power” study on one hand and a possible “over-power” study on another, the difference and similarity between the two trials are first examined by the present invention in view of their primary and secondary endpoints. Motivated by the definition of “recovery” used in ACTT, the present invention then forms a binary endpoint of a properly defined “response”—an idea first suggested in [30] and also listed as one of the three endpoints in a recent Guidance for Industry [37] issued by the US FDA for COVID-19. The present invention then re-analyzes the data from the Chinese remdesivir trial by performing landmark logistic regression analyses with the newly defined binary endpoint. The findings derived from this re-analysis effort should shed some light on the efficacy of remdesivir in the Chinese trial—whether it was really an underpowered study or not, to what extend and on which patient population remdesivir is effective.

### Methods

### Ordinal Scale of COVID-19 Severity and Endpoints

Both the Chinese and the US trials used an ordinal scale of categories to indicate patient's disease severity status at a specific day, which was based on a blueprint of the World Health Organization (WHO) in treating COVID-19 [38]. The Chinese trial used the 6-point scale. NIAID's ACTT used a 7-point scale and then revised to an 8-point scale (revision date: Mar. 20, 2020) [33]. Aside from the reversed order of points, ACTT refined the “Live discharge from hospital” in the Chinese trial scale into two more categories. Furthermore, the 8-point scale in ACTT version #2 refined the point=5 category in version #1 into point 5 and point 6 categories. It is noted that point=5 category of ACTT version #1 corresponds to the Chinese trial's scale point=2 category exactly. These all indicate “mildly severe” status that the patient was hospitalized, but not requiring supplemental oxygen.

ACTT has gone through multiple revision of endpoints. Prior to March 20, the primary endpoint of ACTT was “percentage of subjects reporting each severity rating on the 7-point ordinal scale”; between March 20 and April 20, the primary endpoint was changed to “percentage of subjects reporting each severity rating on the 8-point ordinal scale”. After April 20, the primary endpoint was switched to “time to recovery by Day 29”. Day of recovery is defined as the first day on which the subject satisfies one of the following three categories from the ordinal scale: 1) Hospitalized, not requiring supplemental oxygen—no longer requires ongoing medical care (Point=6); 2) Not hospitalized, limitation on activities and/or requiring home oxygen (Point=7); 3) Not hospitalized, no limitations on activities (Point=8). In a time of pandemic, it is difficult to identify exactly what an appropriate endpoint would be designated, these revisions appeared to be understood and accepted by the regulatory agency [36].

In contrast, the Chinese trial defined the primary endpoint TTCI as “time to a 2-point reduction in patients' admission status on the 6-point ordinal scale, or live discharge from the hospital, whichever came first”. The percentage of subjects reporting each severity rating on the 6-point ordinal scale was a key secondary endpoint. This key secondary endpoint was used by the IDMC to monitor the Chinese trial [30]. Another endpoint was time to a 1-point reduction, which is also included in the NIAID trial as a secondary endpoint.

The endpoint of time to recovery or clinical improvement, whether defined by 1 or 2-point improvement as TTCI in the Chinese trial, or in the NIAID's ACTT, seemed to have escaped the difficulty of “hazard ratio” interpretation and enjoyed a simpler understanding of “median day to response” for clinicians and journalists. However, this kind of time-to-response endpoint has some technical limitations. First, the scores might fluctuate, especially when the scale was refined into more categories. Thus, the “time to response” really meant as time to the first response, ignoring the possibility of sequential worsening on a later day. Second, time-to-improvement does not make clinical sense for patients who died during the study. For the severe COVID-19 cases, the 28-day mortality rate was about 13-14% in the Chinese trial and 8-12% in the NIAID trial. For the dead, TTCI or the time to recovery is infinite or undefined, but has been censored at day 28 or 29. The censoring is obviously an unfair accounting to patients who were alive without reaching the recovery or improvement criterion by the end of the study. The present invention explored the following alternative analysis.

### Alternative Data Analysis for the Chinese Trial

Based on the NIAID trial, in which the “recovery” criterion was defined by reaching categories with point=6, 7, or 8, the present invention sought the corresponding categories in the Chinese trial and determine similarly the “recovery” criterion as reaching the clinical status with point=2 or 1 in the 6-category (reversed) scale. As expressed by clinical experts [33,34], sparing severely ill patients from requiring supplemental oxygen in the pandemic crisis is clinically meaningful to the patients and health-care providers.

The present invention classified each outcome in the Chinese trial a “response” or “non-response” at each assessment day by examining the 6-point scale status: Point=2 or 1 being a response; otherwise a nonresponse. The present invention then analyzed the binary response data with the method of logistic regression. Our analysis is based on the summary data shown in [30] at the last IDMC meeting on Mar. 29, 2020, which is close to the completion of the trial's final data lock on Apr. 1, 2020 reported in [29]. The logistic regression model included the baseline disease status, treatment group, assessment day, treatment by day interaction, and treatment by baseline status interaction. Noticed that this model will obtain the treatment effect adjusted for the baseline status and assessment day in the study. Our main aim is to assess the treatment effect at Day 28 while controlling for baseline status. The present invention also tested the treatment effect at Day 14 to see if there is an early treatment effect 4 days after the 10-day intravenous regimen of remdesivir. Given that the two analyses at the two different days are correlated, the present invention used the Hochberg's step-wise procedure to control the overall type-I error rate [39]: test the hypothesis associated with the smaller p-value against alpha=0.025 and that associated with the larger p-value against alpha=0.05 level. The treatment effect of remdesivir was expressed in terms of odds ratio of response (with 95% confidence interval) relative to the placebo.

### Results

The dataset included 231 patients (153 remdesivir, 78 placebo) for the 6-point ordinal scale at baseline and 225 patients (149 remdesivir, 76 placebo) on Day 28. The baseline score distribution (%) is summarized in Table 5: (0, 0, 81.0, 17.6, 0.7, 0.7) for the remdesivir group and (0, 3.8, 83.3, 11.5, 1.3, 0) for the placebo group, for point=1 (discharged or met discharge criteria) to 6 (death). As seen, majority (81-83%) were point=3 patients, who were hospitalized, required supplemental oxygen (but not NIV/HFNC)—the moderately severe category. About 12-18% were point=4 patients, who were hospitalized and required non-invasive ventilation (NIV) and/or high-flow oxygen therapy (HFNC). Very few in the category 5, who required extracorporeal membrane oxygenation (ECMO) and/or invasive mechanical ventilation (IMV).

TABLE 5
Comparison between the remdesivir group and the placebo group

| | 1 | | 2 | 3 | 4 | 5 | |
|---|---|---|---|---|---|---|---|
| Scale | (Live | | (Mildly | (Moderately | (Critically | (Critically | 6 |
| (Category) | discharge) | | severe) | severe) | severe) | severe) | (Death) |
| Baseline | Remdesivir | 0 | 0 | 124 | 27 | 1 | 1 |
| | n = 153*(%) | (0) | (0) | (81.0) | (17.6) | (0.7) | (0-7) |
| | Placebo | 0 | 3 | 65 | 9 | 1 | 0 |
| | n = 78(%) | (0) | (3.8) | (83.3) | (11.5) | (1.3) | (0) |
| Day 14 | Remdesivir | 45 | 18 | 59 | 12 | 4 | 13 |
| | n = 151(%) | (29.8) | 111.9) | (39.1) | (7.9) | (2.6) | (8.6) |
| | Placebo | 18 | 11 | 27 | 8 | 7 | 7 |
| | n = 78(%) | (23.1) | (14.1) | (34.6) | (10.3) | (9.0). | (9.0) |
| Day 28 | Remdesivir | 99 | 11 | 15 | 2 | 2 | 20 |
| | n = 149(%) | (66.4) | (7.4) | (10.1) | (1.3) | (1.3) | (13.4) |
| | Placebo | 46 | 3 | 12 | 2 | 3 | 10 |
| | n = 76(%) | (60.5) | (3.9) | (15.8) | (2.6) | (3.9) | (13.2) |

*One death occurred prior to receiving treatment excluded from analysis

The proportions of responders (defined as Point≤2), uncontrolled for baseline status, are displayed in FIG. 9 by treatment groups at each study assessment day. The increasing trend of response is obvious for both treatment groups. Table 6 shows the main results from the logistic regression analysis. The response rate was 85% for remdesivir-treated patients with baseline status point=3 (moderately severe category) versus 70% response rate for likewise placebo-treated patients on Day 28 (OR=2.38, P=0.0012). On Day 14, the response rate for these patients was 43% for remdesivir versus 33% for placebo (OR=1.53, P=0.0022). Both were statistically significant with the multiple test adjustment. For patients with baseline status point=4 (critically severe category), which was a much smaller cohort in the study, no similar comparisons were statistically significant, although the placebo group had higher response rate numerically.

TABLE 6
Results from the logistic regression analysis

| | | | | | 95% | | |
|---|---|---|---|---|---|---|---|
| Baseline | | Treatment | Model adjusted | Odds | Confidence | | P- |
| Scale | Day | Group | response rate* | Ratio | Limits | | value |
| 3 | 14 | Placebo | 0.33 | | 0.28 | 0.38 | |
| | | Remdesivir | 0.43 | | 0.39 | 0.46 | |
| | | Remdesivir vs | | 1.53 | 1.17 | 2.01 | 0.0022 |
| | | Placebo | | | | | |
| | 28 | Placebo | 0.70 | | 0.61 | 0.78 | |
| | | Remdesivir | 0.85 | | 0.80 | 0.89 | |
| | | Remdesivir vs | | 2.38 | 1.41 | 4.01 | 0.0012 |
| | | Placebo | | | | | |
| 4 | 14 | Placebo | 0.14 | | 0.07 | 0.25 | |
| | | Remdesivir | 0.07 | | 0.04 | 0.12 | |
| | | Remdesivir vs | | 0.48 | 0.19 | 1.18 | 0.1082 |
| | | Placebo | | | | | |
| | 28 | Placebo | 0.44 | | 0.27 | 0.63 | |
| | | Remdesivir | 0.37 | | 0.25 | 0.50 | |
| | | Remdesivir vs | | 0.74 | 0.29 | 1.89 | 0.5296 |
| | | Placebo | | | | | |

*Logistic regression model includes treatment group, baseline scale, day of assessment, treatment by day interaction, and treatment by baseline interaction.

It is clear that the logistic regression analysis of the binary endpoint provides more statistical power for the data, and shows that the remdesivir in 10-day regimen is effective for moderately severe COVID-19 patients in improving the odds of response by 2.4 folds on Day 28 and 1.5 folds on Day 14 since the start of treatment, with high statistical significance. Thus, the Chinese study was not really “under-powered”, despite of its early end of patient enrollment. But why and how is this logistic regression analysis statistically valid and clinically sound? For these questions, the present invention offers the following points:

The binary endpoint that pools the scale=2 and 1 together as “response” has been suggested by the IDMC prior to the final data analysis as an alternative to the time-to-clinical improvement (TTCI) endpoint [29], and it was recommended by the FDA [36]. It may not have been chosen as the pre-specified primary endpoint as COVID-19 is basically unknown (eg, ACTT made several adaptations regarding endpoints and sample sizes during the course of the trial, as its study title properly prepared), but the binary response is well justifiable. Similarly to oncology phase II trials, a complete response (CR) and partial response (PR) are usually pooled together as “response”, and a rest stable disease (SD) and disease progression (DP) are pooled as “non-response” for an ORR (objective response rate) analysis. The dichotomization of multi-level scale aggregates more events on both sides of “response” versus “non-response”, hence sharpens the comparison and strengthens the signal. This process makes the analysis more powerful than using the original multi-level scale. The landmark analysis at Day 28—the end of follow-up day is also simple and clear for interpretation. On the contrary, the time-to-recovery or TTCI has an intrinsic problem for the dead whose time measure would be infinite or undefined. The binary endpoint also makes sense to clinicians; after all, their decision is always of a binary nature: whether ok to use this drug to treat patient. The binary endpoint is also clinically meaningful because when patients no longer require supplementary oxygen (scale=2) or are discharged from hospital (scale=1), the disease burden is released from the patients and the health-care facilities.

In conclusion, our re-analysis demonstrated that good response rates were achieved with strong statistical significance for remdesivir for the moderately severe patients; valid conclusions can still be made despite the early termination and insufficient sample size. The re-analysis supports the preliminary finding in ACTT that remdesivir is effective, but the present invention qualified that the efficacy applies only to patients whose COVID-19 condition at enrollment was not critically severe, which is the majority of hospitalized patients with COVID-19. the present invention also evidences the decision that remdesivir be available as a part of standard care in the hospital setting in recognition of the urgent need, and agree that the FDA's issuance of EUA is an important step toward developing more effective therapies for all range of COVID-19 patients.

### REFERENCES

[1] Clinical Development Success Rates 2006-2015, BIO Industry Analysis.
[2] Pocock, S. J., (1977). Group sequential methods in the design and analysis of clinical trials. Biometrika, 64, 191-199.
[3] O'Brien, P. C. and Fleming, T. R. (1979). A multiple testing procedure for clinical trials. Biometrics 35, 549-556.
[4] Tsiatis, A. (1982). ‘Repeated significance testing for a general class of statistics used in censored survival analysis’, Journal of the American Statistical Association, 77, 855-861.
[5] Lan, K. K. G., DeMets, D. L. (1983). Discrete sequential boundaries for clinical trials. Biometrika 70:659-663.
[6] Lan, K. K. G. and Wittes, J. (1988). The B-value: A tool for monitoring data. Biometrics 44, 579-585.
[7] Lan, K. K. G. and Demets, D. L (1989). Changing frequency of interim analysis in sequential monitoring, Biometrics, 45, 1017-1020.
[8] Lan, K. K. G., Rosenberger, W. F. and Lachin, J. M. (1993) Use of spending functions for occasional or continuous monitoring of data in clinical trials, Statistics in Medicine, 12, 2219-2231
[9] Wittes, J. and Brittain, E. (1990). The role of internal pilot studies in increasing the efficiency of clinical trials. Statistics in Medicine 9, 65-72.
[10] Shih, W. J. (1992). Sample size reestimation in clinical trials. In Biopharmaceutical Sequential Statistical Applications, K. Peace (ed), 285-301.
[11] Gould, A. L., & Shih, W. J. (1992). Sample size re-estimation without unblinding for normally distributed outcomes with unknown variance.
[12] Herson, J., & Wittes, J. (1993). The Use of Interim Analysis for Sample Size Adjustment. Drug Information Journal, 27(3), 753-760.
[13] Shih, W. J. (2001). Commentary: Sample size re-estimation—Journey for a decade. Statistics in Medicine, 20:515-518.
[14] Bauer, P., & Kohne, K. (1994). Evaluation of Experiments with Adaptive Interim Analyses. Biometrics, 50(4), 1029-1041.
[15] Proschan, M., & Hunsberger, S. (1995). Designed Extension of Studies Based on Conditional Power. Biometrics, 51(4), 1315-1324.
[16] Cui, L., Hung, H. M., Wang, S. J. (1999). Modification of sample size in group sequential clinical trials. Biometrics 55:853-857.
[17] Li, G., Shih, W. J., Xie, T., & Lu, J. (2002). A sample size adjustment procedure for clinical trials based on conditional power. Biostatistics, 3 2, 277-87.
[18] Chen Y H, DeMets D L, Lan K K (2004). Increasing the sample size when the unblinded interim result is promising. Statistics in Medicine, 23:1023-1038.
[19] Posch, M., Koenig, F., Branson, M., Brannath, W., Dunger-Baldauf, C. and Bauer, P. (2005), Testing and estimation in flexible group sequential designs with adaptive treatment selection. Statistics in Medicine, 24: 3697-3714.
[20] Gao P, Ware J H, Mehta C. (2008), Sample size re-estimation for adaptive sequential designs. Journal of Biopharmaceutical Statistics, 18: 1184-1196, 2008
[21] Gao P, Liu L. Y, and Mehta C. (2013). Exact inference for adaptive group sequential designs. Statistics in Medicine, 32, 3991-4005
[22] Bowden, J. and Mander, A. (2014). A review and re-interpretation of a group-sequential approach to sample size re-estimation in two-stage trials. Pharmaceut. Statist., 13: 163-172.
[23] Shih W. J., Li G., Wang Y. (2016) Methods for flexible sample-size design in clinical trials: Likelihood, weighted, dual test, and promising zone approaches. Contemporary Clinical Trials, 47, 40-48.
[24] Michael Proschan, K. K. Gordon Lan and Janet Wittes: Statistical Monitoring of Clinical Trials—A Unified approach, ©2006 Springer Science and Business Media, L.L.C.
[25] Mehta, C. R. and Pocock, S. J. (2011), Adaptive increase in sample size when interim results are promising: A practical guide with examples. Statistics in Medicine, 30: 3267-3284.
[26] Lan, K. K. G., Simon, R., Halperin, M. (1982) Stochastically curtailed tests in long-term clinical trials, Communications in Statistics. Part C: Sequential Analysis, 1:3, 207-219.
[27] Xi, D., Gallo, P., Ohlssen, D. (2017) On the Optimal Timing of Futility Interim Analyses, Statistics in Biopharmaceutical Research, 9:3, 293-301.
[28] Davis, B., Kerr, D., Maguire, M., Sanders, C., Snapinn, S., & Wittes, J. (2018). University of Pennsylvania 10th annual conference on statistical issues in clinical trials: Current issues regarding data and safety monitoring committees in clinical trials (morning panel session). Clinical Trials, 15(4), 335-351.
[29] Wang, Y., et al, Remdesivir in adults with severe COVID-19: a randomised, double-blind, placebo-controlled, multicentre trial, Lancet, Apr. 29, 2020, DOI: 10.1016/S0140-6736(20)31022-9.
[30] Shih, W., Yao, C. and Xie, T. Data Monitoring for the Chinese Clinical Trials of Remdesivir in Treating Patients with COVID-19 During the Pandemic Crisis, Therapeutic Innovation & Regulatory Science, DOI: 10.1007/s43441-020-00159-7.
[31] A Phase 3 Randomized, Double-blind, Placebo-controlled, Multicenter Study to Evaluate the Efficacy and Safety of Remdesivir in Hospitalized Adult Patients With Severe 2019-nCoVRespiratory Disease. PI: Cao Bin. (ClinicalTrials.gov Identifier: NCT04257656)
[32] Norrie J D. Remdesivir for COVID-19: challenges of underpowered studies. Lancet 2020; published Online April 29. https://doi.org/10.1016/S0140-6736(20)31023-0.
[33] A Multicenter, Adaptive, Randomized Blinded Controlled Trial of the Safety and Efficacy of Investigational Therapeutics for the Treatment of COVID-19 in Hospitalized Adults. National Institute of Allergy and Infectious Diseases (NIAID). ClinicalTrials.gov Identifier: NCT04280705.
[34] Beigel J H, Tomashek K M, Dodd L E, et al. Remdesivir for the Treatment of Covid-19 Preliminary Report. N Engl J Med May 22, 2020; DOI: 10.1056/NEJMoa2007764.
[35] Hughes S. Remdesivir Now ‘Standard of Care’ for COVID-19, Fauci Says—Multiple Trials Release Data, Some in Partial Form. Medscape Apr. 29, 2020; https://www.medscape.com/viewarticle/929685
[36] Frellick M. FDA Authorizes Emergency Use of Remdesivir for COVID-19. Medscape May 1, 2020 https://www.medscape.com/viewarticle/929836.
[37] COVID-19: Developing Drugs and Biological Products for Treatment or Prevention, Guidance for Industry, U.S. Department of Health and Human Services, Food and Drug Administration, Center for Drug Evaluation and Research (CDER), Center for Biologics Evaluation and Research (CBER), May 2020.
[38] World Health Organization WHO R&D Blueprint novel Coronavirus: Outline of trial designs for experimental therapeutics, 2020.
[39] Hochberg Y. A sharper Bonferroni procedure for multiple tests of significance. Biometrika 1988; 75:800-802.
[40] Scharfstein, D. O., Tsiatis, A. A., & Robins, J. M. (1997). Semiparametric efficiency and its implication on the design and analysis of group-sequential studies. Journal of the American Statistical Association, 92(440): 1342-1350.

1-15. (canceled)

16. A graphical user interface-based system for monitoring and guiding an ongoing clinical trial on an adjustable and real-time basis, comprising:
a. a clinical trial database for storing information from an ongoing clinical trial, wherein said information comprises a set of subject data that is being continuously updated as said ongoing clinical trial proceeds;
b. a boundary determination module for determining boundaries for a group of regions comprising a favorable region, a hopeful region and an undesirable region, wherein said boundaries are subject to boundary adjustment as said ongoing clinical trial proceeds, wherein each region represents a different level of risk associated with an accumulative effect of said ongoing clinical trial; and
c. a graphical user interface (GUI), operable with said boundary determination module, for displaying a plot of said accumulative effect of said ongoing clinical trial and displaying boundary parameters corresponding to said group of regions, wherein said GUI allows a user to adjust values of boundary parameters in view of said plot, thus generating new boundaries on a real-time basis as said ongoing clinical trial proceeds, wherein said accumulative effect of said ongoing clinical trial is continuously projected onto said plot, thereby monitoring and guiding said ongoing clinical trial on an adjustable and real-time basis, and providing a recommendation, wherein said recommendation is:
1) “early termination for success” if said accumulative effect falls into said successful region;
2) “early termination for futility” if said accumulative effect falls into said futility region;
3) “continuation without modification” if said accumulative effect falls into said favorable region but not said successful region;
4) “continuation with sample size re-estimation” if said accumulative effect falls into said hopeful region; or
5) “continuation with caution” if said accumulative effect falls into said undesirable region but not futility region;
wherein said undesirable region comprises a futility region, and said favorable region comprises a successful region.

17. The system of claim 16, wherein said set of subject data comprises unblinded data or one or more accumulative effects derived from said unblinded data.

18. The system of claim 16, wherein said accumulative effect is one or more statistical scores selected from the group consisting of Score statistics (B value), Wald statistics (Z value), point estimate \hat{θ}, and 95% confidence interval, conditional power (CP), type I error and type II error.

19. The system of claim 16, wherein said boundary parameters have desirable values that are phase- or time-specific.

20. The system of claim 16, wherein the favorable region corresponds to a region where the B value is no less than b₁(t, 1−β); the hopeful region corresponds to a region where the B value is less than b₁(t, 1−β) but no less than b₂(t, Rₘₐₓ); and the undesirable region corresponds to a region wherein the B value is less than b₂(t, Rₘₐₓ); wherein said Rₘₐₓ is a maximum sample size ratio of said ongoing clinical trial at time t.

21. The system of claim 16, wherein said futility region corresponds to a region wherein the B value is no more than b_{f}(t), wherein b_{f}(t) is the threshold value at time t indicating a statistically significant conclusion for futility and said successful region corresponds to a region wherein the B value is no less than Cs, wherein Cs is the threshold value indicating a statistically significant conclusion for success.

22. The system of claim 16, wherein when said ongoing clinical trial falls into said the hopeful region for 10 points consecutively, the system provides a signal indicating necessity to adjust one or more clinical trial parameters of said ongoing clinical trials.

23. A graphical user interface-based system for monitoring and guiding an ongoing clinical trial on an adjustable and real-time basis, comprising:
a. a clinical trial database for storing information from an ongoing clinical trial, wherein said information comprises a set of subject data that is being continuously updated as said ongoing clinical trial proceeds;
b. a boundary determination module for determining boundaries for a group of regions comprising a favorable region, a hopeful region and an undesirable region, wherein said boundaries are subject to boundary adjustment as said ongoing clinical trial proceeds, wherein each region represents a different level of risk associated with an accumulative effect of said ongoing clinical trial;
c. a graphical user interface (GUI), operable with said boundary determination module, for displaying a plot of said accumulative effect of said ongoing clinical trial and displaying boundary parameters corresponding to said group of regions, wherein said GUI allows a user to adjust values of boundary parameters in view of said plot, thus generating new boundaries on a real-time basis as said ongoing clinical trial proceeds, wherein said accumulative effect of said ongoing clinical trial is continuously projected onto said plot, thereby monitoring and guiding said ongoing clinical trial on an adjustable and real-time basis; and
d. a simulation module which conducts simulations in view said set of subject data as accumulated and its trend of said plot, predicts trend and trajectory of said ongoing clinical trial in the future and optionally proposes a clinical trial parameter adjustment by comparing with an initial or existing clinical trial design and assumptions used for said initial or existing clinical design, and said simulations are conducted with a trend analysis or a piecewise linear trend analysis in which different weights are assigned to each piece showing a linear trend.

24. The system of claim 23, wherein said set of subject data comprises unblinded data or one or more accumulative effects derived from said unblinded data.

25. The system of claim 23, wherein said accumulative effect is one or more statistical scores selected from the group consisting of Score statistics (B value), Wald statistics (Z value), point estimate \hat{θ}, and 95% confidence interval, conditional power (CP), type I error and type II error.

26. The system of claim 23, wherein said boundary parameters have desirable values that are phase- or time-specific.

27. The system of claim 23, wherein when said ongoing clinical trial falls into said hopeful region for 10 points consecutively, the system provides a signal indicating necessity to adjust one or more clinical trial parameters of said ongoing clinical trials.

28. The system of claim 23, wherein the favorable region corresponds to a region where the B value is no less than b₁(t, 1−β); the hopeful region corresponds to a region where the B value is less than b₁(t, 1−β) but no less than b₂(t, Rₘₐₓ); and the undesirable region corresponds to a region wherein the B value is less than b₂(t, Rₘₐₓ); wherein said Rₘₐₓ is a maximum sample size ratio of said ongoing clinical trial at time t.

29. The system of claim 23, wherein said futility region corresponds to a region wherein the B value is no more than b_{f}(t), wherein b_{f}(t) is the threshold value at time t indicating a statistically significant conclusion for futility and said successful region corresponds to a region wherein the B value is no less than Cs, wherein Cs is the threshold value indicating a statistically significant conclusion for success.

30. A graphical user interface-based method for monitoring and guiding an ongoing clinical trial on an adjustable and real-time basis, comprising:
a. storing information from an ongoing clinical trial into a clinical trial database, wherein said information comprises a set of subject data that is being continuously updated as said ongoing clinical trial proceeds;
b. mapping boundaries, via a boundary determination module, for a group of regions comprising a successful region, a favorable region, a hopeful region, an undesirable region and a futility region, wherein said boundaries are subject to boundary adjustment as said ongoing clinical trial proceeds, wherein each region represents a different level of risk associated with an accumulative effect of said ongoing clinical trial;
c. conducting said boundary adjustment on a graphical user interface (GUI), wherein said GUI displays a plot of said accumulative effect of said ongoing clinical trial and boundary parameters corresponding to said group of regions, said GUI allows a user to adjust values of said boundary parameters in view of said plot, thus generating new boundaries on a real-time basis as said ongoing clinical trial proceeds,
wherein said accumulative effect of said ongoing clinical trial is continuously projected onto said plot; and
d. providing, via said GUI, a recommendation guiding said ongoing clinical trial, wherein said recommendation is:
1) “early termination for success” if said accumulative effect falls into said successful region;
2) “early termination for futility” if said accumulative effect falls into said futility region;
3) “continuation without modification” if said accumulative effect falls into said favorable region but not said successful region;
4) “continuation with sample size re-estimation” if said accumulative effect falls into said hopeful region; or
5) “continuation with caution” if said accumulative effect falls into said undesirable region but not futility region.

31. A graphical user interface-based method for diagnosing an already completed clinical trial, comprising:
a. sequentially applying information from an already completed clinical trial into a clinical trial database according to time of patient data completion, wherein said information comprises a set of subject data that is being continuously updated;
b. mapping boundaries, via a boundary determination module, for a group of regions comprising a successful region, a favorable region, a hopeful region, an undesirable region and a futility region subject to boundary adjustment as said information is being applied, wherein each region represents a different level of risk associated with an accumulative effect of said clinical trial;
c. conducting said boundary adjustment on a graphical user interface (GUI), wherein said GUI displays a plot of said accumulative effect of said ongoing clinical trial, and boundary parameters corresponding to said group of regions, said GUI allows a user to adjust values of said boundary parameters in view of said plot, thus generating new boundaries assuming said clinical trial was proceeding, wherein said accumulative effect of said clinical trial is continuously projected onto said plot; and
d. providing, via said GUI, a diagnosis of said clinical trial assuming said clinical trial was proceeding, wherein said diagnosis is:
1) “early termination for success” if said accumulative effect falls into said successful region;
2) “early termination for futility” if said accumulative effect falls into said futility region;
3) “continuation without modification” if said accumulative effect falls into said favorable region but not said successful region;
4) “continuation with sample size re-estimation” if said accumulative effect falls into said hopeful region; or
5) “continuation with caution” if said accumulative effect falls into said undesirable region but not futility region.
